(19) United States
(12) Patent Application Publication
(10) Pub. No.: US 2014/0227694 A1
(43) Pub. Date: Aug. 14, 2014

(54) EPIGENETIC MARKERS OF PLURIPOTENCY

(71) Applicant: ZYMO RESEARCH CORPORATION, Irvine, CA (US)

(72) Inventors: Jill PETRISKO, Irvine, CA (US); Lam NGUYEN, Irvine, CA (US); Manuel KRISPIN, Irvine, CA (US)

(73) Assignee: ZYMO RESEARCH CORPORATION, Irvine, CA (US)

(21) Appl. No.: 14/176,226

(22) Filed: Feb. 10, 2014

Related U.S. Application Data
(60) Provisional application No. 61/762,672, filed on Feb. 8, 2013.

(51) Int. Cl. C12Q 1/68

(52) U.S. Cl. CPC C12Q 1/6881
USPC 435/6.11; 536/24.33

(57) ABSTRACT

Epigenetic methods for assessing pluripotency of a cell population, such as a stem cell culture are provided. For example, pluripotency can be assessed by determining DNA methylation status at the RAB25, NANOG, PTPN6, MGMT, GBP3 and/or LYST gene regions. Kits and reagents for testing cells are likewise provided.

This application claims the benefit of U.S. Provisional Patent Application No. 61/762,672, filed Feb. 8, 2013, the entirety of which is incorporated herein by reference.

### INCORPORATION OF SEQUENCE LISTING

The sequence listing that is contained in the file named “ZYMO.P0019US_ST25.txt”, which is 8 KB (as measured in Microsoft Windows®) and was created on Feb. 3, 2014, is filed herewith by electronic submission and is incorporated by reference herein.

### BACKGROUND OF THE INVENTION

1. Field of the Invention

The present invention relates generally to the field of molecular biology, medicine and epigenetics. More particularly, it concerns methods for determining pluripotency in cells human embryonic and induced pluripotent stem cells.

2. Description of Related Art

The ability to characterize the pluripotent state of an embryonic or induced pluripotent stem (iPS) cell is paramount to the field of stem cell research. Changes in gene expression and epigenetic profiles can occur during passaging of stem cells which may have profound effects on the outcome of later experiments or differentiation protocols. To date the NIH criteria for defining a pluripotent stem cell include the ability to form a teratoma; unlimited self-renewal in culture; expression of OCT4/POUF5F1, SOX2, and NANOG; expression of specific cell surface markers such as SSEA-3, SSEA-4, TRA-1-60, TRA-1-80; the formation of embryoid bodies; a specific pattern of gene expression assayed by whole genome profiling. However, standards for determining the pluripotency of human embryonic stem cell lines are still being investigated and there remains a need for a rapid and accurate method test to assess and monitor pluripotency.

### SUMMARY OF THE INVENTION

In a first embodiment there is provided a method for assessing pluripotency (or differentiation status) in a cell population by assessing the methylation status of specific gene regions in genomic DNA of the cell population. For instance, a method is provided for assessing pluripotency in a cell population comprising (a) obtaining a nucleic acid sample from the cell population; and (b) determining the DNA methylation status at 2 or more gene regions selected from the group consisting of RAB25, NANOG, PTPN6, MGMT, GBP3 and LYST. Thus, in some aspects, an increased level of DNA methylation relative to a reference at MGMT, GBP3 or LYST; or a decreased level of DNA methylation relative to a reference at RAB25, NANOG or PTPN6 indicates that the cell population comprises pluripotent cells (e.g., embryonic stem cells, or iPS cells). Conversely, a decreased level of DNA methylation relative to a reference at MGMT, GBP3 or LYST; or an increased level of DNA methylation relative to a reference at RAB25, NANOG or PTPN6 can indicate that the cell population comprises differentiated cells.

In certain aspects, a method of the embodiments further comprises (c) identifying the cell population as comprising pluripotent cells or as comprising differentiated (or partially differentiated) cells. For example, a cell population can be identified as comprising pluripotent cells if the cells are determined to have an increased level of DNA methylation relative to a reference at MGMT, GBP3 or LYST; or a decreased level of DNA methylation relative to a reference at RAB25, NANOG or PTPN6. Conversely, the cell population can be identified as comprising differentiated cells if the cells are determined to have an increased level of DNA methylation relative to a reference at RAB25, NANOG or PTPN6; or a decreased level of DNA methylation at MGMT, GBP3 or LYST. Thus, in some aspects, a method of the embodiments comprises identifying a cell or cell population as substantially pluripotent if the cell(s) are determined to have a level of methylation of between about 0-10% at RAB25, 0-5% at NANOG, 0-10% at PTPN6, 10-40% at MGMT, 20-60% at GBP3 and/or 50-100% at LYST. Conversely, in some aspects, a method of the embodiments comprises identifying a cell or cell population as substantially differentiated if the cell(s) are determined to have a level of methylation of between about 20-80% at RAB25, 5-30% at NANOG, 5-50% at PTPN6, 0-10% at MGMT, 0-20% at GBP3 and/or 0-40% at LYST. Furthermore, DNA methylation differences can be communicated in different formats (e.g., converted to Methylation scores). Therefore, in some aspects, the percent methylation values disclosed herein (see, e.g., FIG. 7) maybe representative of the methodology used, e.g., a method such as that disclosed in the Examples (See V.). A panel of markers of the embodiments may also be assayed on other platforms where similar but numerically slightly different values would be expected. Nonetheless, significant methylation differences in the markers between the sample cells (i.e., pluripotent vs. differentiated) would allow accurate assessment of the pluripotency of sample cells.

In certain aspects, identifying the cell population as comprising pluripotent or differentiated cells in accordance with the embodiments comprises reporting whether the cell population comprises such cells. For example, in some aspects, reporting comprises providing an electronic, written or oral report.

In a further aspect, a method of the embodiments is further defined as a method for monitoring differentiation status in cell populations. Accordingly, in certain aspects, a method comprises (a) obtaining a plurality of DNA samples (i.e., two or more DNA samples) from cell populations at different time points or under different treatment conditions; and (b) determining the DNA methylation status at 2 or more gene regions selected from the group consisting of RAB25, NANOG, PTPN6, MGMT, GBP3 and LYST in the plurality of DNA samples, wherein an increased level of DNA methylation relative to a reference at MGMT, GBP3 or LYST; or a decreased level of DNA methylation relative to a reference at RAB25, NANOG or PTPN6 indicates that the cell population comprises greater pluripotency at a given time point or under a given treatment condition. Conversely, cases where a decreased level of DNA methylation relative to a reference at MGMT, GBP3 or LYST; or an increased level of DNA methylation relative to a reference at RAB25, NANOG or PTPN6 is observed would indicate that the cell population comprises less pluripotency at a given time point or under a given treatment condition. For instance, assessing a differentiation status can comprise assessing the stage of differentiation of cell population, such as whether the cell population is pluripotent, multipotent, partially differentiated or terminally differentiated.

In some aspects a method of the embodiments further comprises determining the DNA methylation status at least one of the gene regions selected from RAB25, NANOG and PTPN6 and at least one of the gene regions selected from MGMT, GBP3 and LYST. For example, a method can comprise determining the DNA methylation status at MGMT and, one, two, three, four or all five, of the gene regions selected from RAB25, NANOG, PTPN6, GBP3 and LYST. In still further aspects, a method comprises determining the DNA methylation status at 3, 4, 5 or all six of the MGMT RAB25, NANOG, PTPN6, GBP3 or LYST gene regions. In certain aspects, a method involves determining the methylation status of a group of gene regions consisting essentially of MGMT RAB25, NANOG, PTPN6, GBP3 and LYST. In yet a further aspect, a method of the embodiments comprises determine the methylation status at a CpG position provided in SEQ ID NO: 13, 14, 15, 16, 17 and/or 18.

In yet further embodiment, a method is provided for assessing pluripotency in a cell population comprising (a) obtaining a nucleic acid sample from the cell population; and (b) determining the DNA methylation status at 2 or more gene regions selected from the group consisting of RAB25, NANOG, PTPN6, MGMT, GBP3, LYST, ARMC7, 5LOX, SOX2, OCT4, SALL4, SP100, UBEIL, EPHA1, REX1, GATA6, BMP4, and NESTIN. Thus, in certain aspects, a method of the embodiments comprises determining the methylation status at 5, 6, 7, 8, 9, 10 or more gene region. In certain aspects, a method of the embodiments comprises determining the DNA methylation status at RAB25, NANOG, PTPN6, MGMT, GBP3, LYST and at least one, two or three additional gene regions selected from the group consisting of ARMC7, 5LOX, SOX2, OCT4, SALL4, SP100, UBEIL, EPHA1, REX1, GATA6, BMP4, and NESTIN. For example, a cell population comprising pluripotent cells could comprise increased methylation in the SP100 and/or UBEIL gene regions. In some aspects, a cell population comprising pluripotent cells exhibits decreased methylation of ARMC7, 5LOX, SOX2, OCT4, SALL4, EPHA1 and/or REX1 gene regions relative to a reference level.

In a further embodiment there is provided a method for assessing pluripotency in a cell population comprising (a) obtaining a DNA sample from the cell population; (b) identifying two or more genomic amplification intervals, each interval comprising at least one CpG position within the recognition sequence of a methylation sensitive restriction enzyme (MSRE), where the CpG position is subject to differential methylation during cell differentiation; (c) amplifying the two or more genomic amplification intervals in the presence and absence of the MSRE; and (d) quantifying the amount of amplification product corresponding to each interval (with and without MSRE treatment) to determine the proportion of DNA methylation in the two or more genomic amplification intervals, thereby assessing pluripotency in the cell population. For example, assessing pluripotency in accordance with the instant embodiment can comprise comparing the proportion of DNA methylation in the two or more genomic amplification intervals with a reference level of DNA methylation (e.g., for differentiated cells or for stem cells) to assess the pluripotency of the cell population. In certain aspects, a method further comprises identifying and amplifying 3, 4, 5, 6 or more genomic amplification intervals. For example, one or more the genomic amplification intervals can comprise a CpG position provided in SEQ ID NO: 13, 14, 15, 16, 17 and/or 18. Thus, in some aspects, methods of the embodiments may be used to monitor or assess the pluripotency of a candidate induced pluripotent stem (iPS) cell or a population thereof.

In some aspects a method of the embodiments is further defined a method for monitoring the differentiation status in cell populations. For example, such a method can comprise the steps of (a) obtaining a plurality of DNA samples from the cell populations at different time points or under different treatment conditions; (b) identifying two or more genomic amplification intervals, each interval comprising at least one CpG position within the recognition sequence of a MSRE, where the CpG position is subject to differential methylation during cell differentiation; (c) amplifying the two or more genomic amplification intervals in the presence and absence of the MSRE; and (d) quantifying the amount of amplification product to determine the proportion of DNA methylation in the two or more genomic amplification intervals in the cell populations at different time points or under different treatment conditions, thereby assessing the differentiation status of the cell populations. For instance, assessing a differentiation status can comprise assessing the stage of differentiation of cell population, such as whether the cell population comprises pluripotent, multipotent, partially differentiated or terminally differentiated cells.

In a further embodiment a method of monitoring the differentiation status of cell population is provided comprising (a) exposing the cell population to at least a first treatment and (b) assessing pluripotency of the cell population by a method of the embodiments. Thus, in certain aspects, a method of the embodiments can be used to determine the effect of two three, four or more treatment conditions on the pluripotency of differentiation status of a cell population.

Certain aspects of the embodiments concern cell populations that are subjected to different treatment conditions or from which samples are obtained at different time points. For example, in some aspects, a method of the embodiments can be used to verify the pluripotency of stem cells or iPS by periodically obtaining samples from the cells and assessing pluripotency. In further aspects, a method can comprise monitoring a differentiation protocol to assess the progress of differentiation by analyzing cell samples in accordance with the embodiments.

Certain aspects of the embodiments concern a population cells, such as a population mammalian cells. In some aspects, the cells are murine, canine, feline, equine, bovine, porcine or rat cells. In preferred aspects, the cells are human cells. Cell populations, in some aspects, comprise primary cells (e.g., primary fibroblast cells) in a culture of primary cells or cell lines. In certain aspects, a cell population comprises stem cells (e.g., embryonic stem cells), iPS cells or partially or terminally differentiated cells. In some aspects, the cell population can be a population of cultured cells. In further aspects, cell population is an in vivo cell population.

As detailed above, in certain aspects, methods are provided for assessing pluripotency in a cell population. In further aspects, a method comprises using the cell population in a further protocol if the cell population is determined to comprise pluripotent cells (or in some cases if the cell population is determined to comprise differentiated cells). In further aspects, a method can comprise discarding the cell population if the cell population is determined to comprise differentiated cells.

Aspects of the embodiments concern determining DNA methylation status at a particular CpG position or at a particular locus in genomic DNA. In some aspects, methylation status is determined by performing a method selected from the group consisting of methylation specific PCR (MSP), real-time methylation specific PCR, methylation-sensitive single-strand conformation analysis (MS-SSCA), quantitative methylation specific PCR (QMSP), PCR using a methylated DNA-specific binding protein, high resolution melting analysis (HRM), methylation-sensitive single-nucleotide primer extension (MS-SnuPE), base-specific cleavage/MALDI-TOF, PCR, real-time PCR, Combined Bisulfite Restriction Analysis (COBRA), methylated DNA immunoprecipitation (MeDIP), a microarray-based method, pyrosequencing, and bisulfite sequencing. For example, determining DNA methylation status can comprise performing methylation specific PCR, real-time methylation specific PCR, QMSP, or bisulfite sequencing. Thus, in some aspects, a method of the embodiment comprises treating nucleic acid in the sample with bisulfite. In further aspects, a method of the embodiments does not comprise treating a sample with bisulfite. For example, in some cases, DNA methylation is determined by MSRE-PCR, see, e.g., PCT Patent Application No. WO 2011/109529, incorporated herein by reference in its entirety. In yet further aspects, determining a methylation status in accordance with the embodiments comprises determining a hydroxymethylation status.

In certain aspects, methods are provided for quantitating the proportion of methylated DNA based on the amount of DNA amplification, such methods are further detailed below. In one aspect, for instance, the proportion of DNA methylation is determined from a change in the cycle threshold (Ct) value obtained from the DNA amplification as compared to an amplification standard. For example, in one embodiment wherein the MSRE or MSRE mixture exhibits reduced cleavage in the presence of DNA methylation that overlaps the enzyme recognition site(s) methylation percent=100×2^{-ΔCt} where ΔCt=Ct obtained from a sample incubated with active MSRE minus the Ct obtained from a sample incubated in a reaction mixture without active MSRE. In embodiments wherein the MSRE or MSRE mixture exhibits increased cleavage in the presence of DNA methylation that overlaps the enzyme recognition site(s) methylation (%)=100−(100×2^{-ΔCt}).

In certain aspects, a method for quantifying site-specific DNA methylation prevalence in a genomic DNA sample comprises (i) digesting a portion (e.g., half) of the DNA sample with MSRE to specifically cleave methylated or non-methylated DNA; (ii) incubating another portion (e.g., the second half) of the DNA sample with inactivated MSRE (or in the absence of an MSRE); (iii) amplifying the MSRE-treated DNA from both samples using a DNA polymerase and oligonucleotide primers in the presence of an oligonucleotide probe or dye to produce amplified samples; and (iv) determining the methylation status be measuring Ct values for the amplified samples. Quantification of site-specific DNA methylation may be accomplished, for example, by comparing the Ct values obtained from the samples to established Ct values correlated to percent DNA methylation (see, e.g., Livak and Schmittgen 2001, incorporated herein by reference).

In some aspects, determining a methylation status at a gene region or in an amplification interval comprises determining an average proportion of methylation in a gene region or in the interval. In further aspects, a method comprises determining which of the CpG position in the gene region or in the interval are methylated or the proportion of methylation at a given CpG position. For example, a method of the embodiments can comprise quantifying the proportion of methylation at one or more CpG positions in a gene region or amplification interval. In some specific aspects, a method comprises quantifying the proportion of methylation at one or more CpG positions provided as provided in SEQ ID NO: 13, 14, 15, 16, 17 and/or 18. In other specific aspects amplification intervals comprise at least one CpG position provided in gene regions selected from the group consisting of RAB25, NANOG, PTPN6, MGMT, GBP3 and LYST. For example the step of assessing pluripotency in the cell population may comprise finding an increased proportion of DNA methylation relative to a reference at MGMT, GBP3 or LYST; or a decreased proportion of DNA methylation relative to a reference at RAB25, NANOG or PTPN6 indicating that the cell population comprises pluripotent cells, or conversely, finding an increased proportion of DNA methylation relative to a reference at RAB25, NANOG or PTPN6; or a decreased proportion of DNA methylation relative to a reference at MGMT, GBP3 or LYST indicating that the cell population comprises differentiated cells.

Thus, in further aspects, a method of the embodiments comprises use of at least a first pair oligonucleotide probes that binds to a DNA sequence in a sample (e.g., flanking an amplification interval). In further embodiments, oligonucleotide primers or probes comprise a label. Alternatively or additionally, a method comprises use of a label that binds to double stranded DNA, such as SYBR® Green, a SYTO dye (e.g., SYTO® 9) or free nucleotides that are labeled. Useful labels include, but are not limited to, fluorescent labels, radioactive labels, sequence labels, enzymatic labels and affinity labels. The presence of labeled (e.g., fluorescently labeled) continuants in the reaction mixture may be used to facilitate detection and quantification of DNA amplification.

In some specific aspects, methods of the embodiments comprise use of an MSRE. Such enzymes may be naturally occurring or engineered recombinant enzymes. Examples of MSREs for use according to the embodiments include, but are not limited to, AccII, AciI, HpaII, HinP1I, HpyCH4IV, AatII, AclI, AfeI, AgeI, AscI, AsiSI, AvaI, BmgBI, BsaAI, BsaHI, BsiEI, BsiWI, BsmBI, BspDI, BstBI, ClaI, EagI, FauI, FnuDII, FseI, FspI, HaeII, HgaI, HhaI, Hpy99I, KasI, MluI, NaeI, NarI, NgoMIV, NotI, NruI, PaeR7I, PmlI, PvuI, RsrII, SacII, SalI, SfoI, SgrAI, SmaI, SnaBI or ZraI. In certain cases, the MSRE is an endonuclease which recognizes a 4-base-pair sequence (e.g., a 4-based pair sequence comprising a CpG dinucleotide sequence). For example, an AciI, AccII, HpaII, Hinp1I or HpyCH4IV enzyme or a combination thereof can be used. In a further aspect, a restriction endonuclease comprises an enzyme having increased activity on methylated DNA substrates (e.g., a methylation-dependent endonuclease). Such enzymes may be naturally occurring or engineered recombinant enzymes. Examples of such MSREs for use according to the embodiments include, but are not limited to, BisI, GlaI, McrBC or a mixture thereof.

Certain aspects of the embodiments concern comparing methylation status to the methylation status of a reference. Such a reference may be a reference sample that subjected to methylation analysis or may be reference level, such as a level provided in a chart or a table. As detailed herein a reference level refers to a methylation level corresponding to a differentiated cell, such as a terminally differentiated cell (e.g., a somatic cell). For example, a reference level can correspond to an average level of methylation from somatic cells. A skilled artisan will recognize, however, that methods of the embodiments can alternatively or additional be practiced using a methylation reference level corresponding to methylation level in stem cells or iPS cells.

In some aspects a method of the embodiment concerns obtaining a nucleic acid sample. In certain cases a sample can be directly obtained from cells by extracting the DNA (e.g., from a preparation of tissue culture cells). Thus, in some cases a sample of in vivo cells is obtained from a subject, such as a human subject. In other aspects, a cell or nucleic acid sample is obtained from a third party.

In still a further embodiment a kit is provided comprising a sealed container comprising primers designed to amplify regions including potential DNA methylation sites. For example, oligonucleotide primers can comprise primer to amplify intervals of two gene regions selected from the group consisting of RAB25, NANOG, PTPN6, MGMT, GBP3 and LYST. For example, primers or probes designed to detect methylation in at least 3, 4, 5 or 6 of said gene regions can be included in a kit. In some specific aspects a kit comprises a primer pair that can amplify intervals from RAB25, NANOG, PTPN6, MGMT, GBP3 and LYST. Examples of primers for use in kits of the embodiments include those that can amplify a sequence including one of the CpG position provided in SEQ ID NO: 13, 14, 15, 16, 17 and/or 18. In some specific aspects, a kit can comprise one or more of the primers provided as SEQ ID NO: 1-12.

In still further aspects a kit of the embodiments further comprises one or more reagent for PCR, real-time PCR, or bisulfite sequencing. In some aspects, a kit comprises at least a first methylation sensitive endonuclease. Further elements and reagents that may be included in a kit of the embodiments include, without limitation, dyes (e.g., fluorescent dyes), RNAase, DNA extraction reagents, a microtiter plate or reaction tubes, reference or control samples and instructions (e.g., including a table or chart of methylation reference levels).

Other embodiments of the invention are discussed throughout this application. Any embodiment discussed with respect to one aspect of the invention applies to other aspects of the invention as well and vice versa. The embodiments in the Example section are understood to be embodiments of the invention that are applicable to all aspects of the invention.

It is specifically contemplated that an individual component or element of a list may be specifically included or excluded from the claimed invention.

It is contemplated that any embodiment discussed herein can be implemented with respect to any method or composition of the invention, and vice versa. Furthermore, compositions and kits of the invention can be used to achieve methods of the invention.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: Biological significance of the pluripotent stem cell epigenetic markers with embryonic cell lines, differentiated cells, and standards. Methylation percentages for each of the six gene regions: RAB25, NANOG, PTPN6, MGMT, GBP3, and LYST show that the five human embryonic stem cell lines ES-017, ES-035, ES-049, ES-051, ES-053, have different methylation percentages than that of the one differentiated cell line. The fully non-methylated and methylated genomic DNA standards (Zymo Research Corp.) are shown as controls. Graphed data for each gene region indicates results, from left to right for, ES-017, ES-035, ES-049, ES-051, ES-053, differentiated cells, non-methylated DNA or methylated DNA.

FIG. 2: Test for assay performance using correlation of actual methylation percentages with calculated methylation percentages. Assay performance: results of the OneStep qMethyl™ Panel closely correlate with actual DNA methylation percent values. A standard curve using NANOG, RAB25, PTPN6, MGMT, GBP3, and LYST primer sets from the OneStep qMethyl™ Panel was performed with 0%, 25%, 50%, 75%, and 100% methylated DNA from mixtures of human non-methylated and methylated DNA. DNA samples were assayed in triplicates using the ABI™ 7500 series.

FIG. 3: Test for assay sensitivity. Differentiated DNA was spiked into stem cell DNA from the human embryonic stem cell line ESI-035 at 0, 0.1, 1, 10, and 100% and the response in each gene was measured. The lowest amount of differentiated DNA that could be detected using the OneStep qMethyl™ Human Pluripotent Stem Cell Panel I protocol was 1% in the NANOG gene.

FIG. 4: Schematics A and B illustrate the sample workflow of methylation detection for non-methylated and methylated DNA regions. In both cases the DNA is divided in two parts; a Test Reaction and a Reference Reaction. Test Reaction samples are MSRE digested while the Reference Reaction samples are not (mock digested). Following digestion, DNA from both samples is used for real-time PCR. The white lollipops in the image represent unmethylated cytosines and black lollipops methylated cytosines in a CpG dinucleotide context. Following real-time PCR, amplification plots (C and D) demonstrate non-methylated DNA exhibits large differences in the Ct values for Test and Reference Reactions (C) while highly methylated DNA samples exhibit little difference (D). These data can be used as the basis for methylation determination in accordance with the embodiments.

FIG. 5A-B: Example arrangements of assays of the embodiments in a multi-well or microtiter plate format. An example assay set-up of FIG. 5A is detailed in Example 3 (shaded wells indicate non-methylated control DNA). An example assay set-up of FIG. 5B is detailed in Example 4 (shaded wells indicate non-methylated control DNA; open wells are human stem cell DNA; striped wells are human differentiated cell DNA).

FIG. 6: Graph shows results of an example assay detailed in Example 4. Human differentiated DNA (grey bars) and human stem cell DNA (white bars) show different DNA methylation percentages for RAB25, NANOG, PTPN6, MGMT, GBP3, and LYST.

FIG. 7: Graph shows results of an additional assay of the embodiments detailed in Example 4. Human adult dermal fibroblast DNA (light grey bars) and human stem cell DNA (white bars) show different DNA methylation percentages for RAB25, NANOG, PTPN6, MGMT, GBP3, and LYST.

### DESCRIPTION OF ILLUSTRATIVE EMBODIMENTS

### I. The Present Invention

The ability to characterize the pluripotent state of an embryonic or induced pluripotent stem cell is paramount to the field of stem cell research. Changes in gene expression and epigenetic profiles can occur during passaging of stem cells which may have profound effects on the outcome of an experiment. According to the National Institute of Health (NIH) standards for determining the pluripotency of human embryonic stem cell lines are still being investigated. Many methods are available for researchers to assess the pluripotent state of embryonic and induced pluripotent stem cells lines, but these methods are typically cumbersome and expensive. For example, DNA methylation profiles based upon Reduced Representative Bisulfite Sequencing (RRBS) and gene expression profiles have been established as a “scorecard” for certain stem cells lines (Bock et al. 2011). PluriTest, an open access bioinformatics assay is another assessment of pluripotency based upon microarray gene expression profiling (Muller et al. 2011). However, information from such studies can be difficult to interpret and may not provide a direct and definitive assessment the differentiation status of cells. Accordingly, there remains a need for efficient methods to assess the pluripotency of cell populations.

Embodiments of the invention address this need by providing methods to definitively assess pluripotency in cells by determining the methylation status of key gene regions in genomic DNA. In particular, studies herein demonstrate that hyper- and hypomethylation in key genomic regions is can be used to accurately discern differentiated cells from pluripotent cells. Rather than requiring time consuming an expensive whole genome analysis, accurate assessment can be accomplished by quantification and methylation at a finite number of positions. Moreover, an assay is provided that quantitatively assesses methylation status at these key genomic positions and, which is highly reproducible. Accordingly, assays provided here can be used to very quickly (and inexpensively) assess the quality of pluripotent cell populations, assess the effectiveness of differentiation protocols or to monitor the progress of a differentiation protocol.

Thus, in one embodiment a real-time PCR based pluripotent stem cell panel is provided (The OneStep qMethyl Human Pluripotent Stem Cell Panel I) that allows researchers (or clinicians) to assess the pluripotent state of an embryonic stem cell or iPS cell population. In such an embodiment bisulfite conversion of DNA is not required. The panel is based upon a specific epigenetic profile of six key genes: RAB25, NANOG, PTPN6, MGMT, GBP3, and LYST. These gene regions have been established to show differential methylation among human embryonic stem cells, induced pluripotent stem cells, and differentiated cells. The panel serves as an additional test to assess the pluripotent epigenetic state of an embryonic cell line or induced pluripotent cell line. One benefit of this panel is that it is accessible to a large number of researchers due to its real-time PCR based platform, unlike unwieldy RRBS and large gene expression profiling methods that require knowledge of bioinformatics. Results from the panel provided here can be easily and quickly interpreted and used for determining methylation status in specific gene regions and thus pluripotency of cells.

The studies presented herein show that, when the methylation percentages of these six genes regions are used in combination they represent an efficient way to discern a pluripotent stem cell epigenetic pattern from that of a differentiated cell epigenetic pattern. Studies show that the methylation patterns of RAB25, NANOG, PTPN6, MGMT, GBP3, and LYST are stable over multiple passages, thereby making this test a valuable resource for laboratories interested in quality control of their human embryonic and induced pluripotent stem cell lines.

### II. Definitions

As used herein the term “gene region” refers to a region of genomic DNA associated with a given gene. For example, the region can be defined by a particular gene (such as protein coding sequence exons, intervening introns and associated expression control sequences) and its flanking sequence. Thus, in some aspects, gene regions are defined by the regions encoding the RAB25, NANOG, PTPN6, MGMT, GBP3 and LYST genes. It is, however, recognized in the art that methylation in a particular region (e.g., at a given CpG position or in a amplification interval) is generally indicative of the methylation status at proximal genomic sites. Accordingly, determining a methylation status of a gene region can comprise determining a methylation status at a site or sites within or flanking about 10 bp to 50 bp, about 50 to 100 bp, about 100 bp to 200 bp, about 200 bp to 300 bp, about 300 to 400 bp, about 400 bp to 500 bp, about 500 bp to 600 bp, about 600 to 700 bp, about 700 bp to 800 bp, about 800 to 900 bp, 900 bp to 1 kb, about 1 kb to 2 kb, about 2 kb to 5 kb, or more of a named gene, or CpG position.

As used herein the term “genomic amplification interval” refers to a region of genomic DNA that can be amplified by PCR. As used herein an amplification interval comprises at least one CpG position that is a potential site of methylation. In some cases, the amplification interval comprises 2, 3, 4 or more potential sites of CpG methylation (e.g., wherein the CpG is in a sequence recognized by an MSRE). In general, an amplification interval is less than about 1,200 bp, such as between about 50 bp and 100, 200, 300, 400 or 500 bp. In preferred aspects the amplification interval is between about 100 and 400 bp.

As used herein “determining a methylation status” for an indicated gene means determining whether one of more position in the DNA of the gene is methylated (or hydroxymethylated). Thus, in certain aspects, determining a methylation status for a gene comprises determining the methylation status at least 1, 2, 3, 4, 5, 6, 7, 8, 9, 10, 15, 20, 25, 30, 35, 40, 45, 50 or more sites of potential DNA methylation.

As used herein, a “methylation sensitive restriction enzyme” or “MSRE” is a restriction endonuclease that includes CG as part of its recognition site and has altered activity when the C is methylated as compared to when the C is not methylated (e.g., Sma I). Non-limiting examples of methylation sensitive restriction endonucleases include MspI, HpaII, BssHII, BstUI, SacII and EagI and Nod. An “isoschizomer” of a methylation sensitive restriction endonuclease is a restriction endonuclease that recognizes the same recognition site as a methylation sensitive restriction endonuclease but cleaves both methylated CGs and unmethylated CGs, such as for example, MspI.

As used herein the “hypermethylation” indicates an increase in the presence of methylation in a sample relative to a reference level. For example, hypermethylation can refer to an increased number of methylated positions in a region of DNA or an increased proportion of DNA molecules in a sample that comprise methylation at a particular position or in a region of DNA sequence.

As used herein the “hypomethylation” indicates decrease in the presence of methylation in a sample relative to a reference level. For example, hypomethylation can refer to a decreased number of methylated positions in a region of DNA or decreased proportion of DNA molecules in a sample that comprise methylation at a particular position or in a region of DNA sequence.

The terms “inhibiting,” “reducing,” or “prevention,” or any variation of these terms, when used in the claims and/or the specification includes any measurable decrease or complete inhibition to achieve a desired result.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

### III. Detection of DNA Methylation

In one aspect, methods of the embodiments concern determining DNA methylation status at plurality of gene regions to determine pluripotency of a cell population. In certain aspects, hypomethylation or hypermethylation in one, two, three, four, five, or all six of RAB25, NANOG, PTPN6, MGMT, GBP3 or LYST gene regions can be used to assess the pluripotency of cells.

Methylation typically occurs in a CpG containing nucleic acid. The CpG containing nucleic acid may be present in, e.g., in a CpG island, a CpG doublet, a promoter, an intron, or an exon. For instance, in the gene regions provided herein the potential methylation sites encompass the promoter/enhancer regions of the indicated genes. Thus, the regions can begin upstream of a gene promoter and extend downstream into the transcribed region. In certain specific aspects, DNA methylation is determined one or more of the regions of the RAB25, NANOG, PTPN6, MGMT, GBP3 or LYST genes.

A variety of methods for detecting the presence, absence, or amount of methylation in a gene are known in the art and may be used to evaluate the methylation status of one or more genes as described herein. For example, allele specific primer extension (ASPE), methylation sensitive restriction enzyme (MSRE) PCR, Real-Time MSRE, methylation specific PCR (MSP), real-time methylation specific PCR, methylation-sensitive single-strand conformation analysis (MS-SSCA), quantitative methylation specific PCR (QMSP), PCR using a methylated DNA-specific binding protein, high resolution melting analysis (HRM), methylation-sensitive single-nucleotide primer extension (MS-SnuPE), base-specific cleavage/MALDI-TOF, PCR, real-time PCR, Combined Bisulfite Restriction Analysis (COBRA), methylated DNA immunoprecipitation (MeDIP), a microarray-based method, pyrosequencing, or bisulfite sequencing may be used to detect and/or quantify methylation in a locus.

Some methods for detecting the presence, absence, or amount of methylation involve exposing genomic DNA to bisulfite. Exposure of DNA, such as genomic DNA, to bisulfite does not affect methylated cytosine in a CpG, while unmethylated cytosines are changed to uracil. Thus, many methods for detecting methylation of a gene involve the treatment of DNA with bisulfite, and subsequent analysis of the resulting nucleotide. Techniques for bisulfate conversion of DNA are well known in the art and commercial kits that provide optimized conversion efficiency are available (e.g., the EZ DNA Methylation-Startup™ kit available from Zymo Research Corp., Irvine, Calif.). Various methods for detecting methylation are disclosed, e.g., in PCT Pub. No. WO/2010/114821; PCT Pub. No. WO/2011/109529; U.S. 2011/0046009, U.S. 2010/0304992, U.S. Pat. No. 5,786,146; Fraga, 2002; El-Maarri, 2003; Laird, 2003; and Callinan, 2006, which are incorporated herein by reference in their entirety

A. Allele Specific Primer Extension (ASPE)

Allele specific primer extension (ASPE) is a method used to interrogate a single nucleotide polymorphism (SNP) at the 3′ end of a preamplified DNA sequence which can be used for methylation analysis. This is done by heat denaturing the preamplified DNA sequence to obtain a single stranded template. The heat is then lowered to a temperature that allows annealing of a primer to either the sense or anti-sense strand of the template. This primer contains a nucleotide at the 3′ end of its sequence that either is, or is not, complimentary to the nucleotide in the template. If the nucleotide is complimentary, stand extension using a proofreading DNA polymerase occurs and forms a double stranded product. If the nucleotide is not complimentary, strand extension does not occur. Strand extension is then quantified using fluorescence. Extension of the product and a high level of fluorescence indicate a match for the nucleotide of interest. No extension and low fluorescence indicates a mismatch for the nucleotide of interest.

B. Methylation Specific PCR (MSP)

Methylation specific PCR typically utilizes bisulfite treatment of a nucleic acid to detect methylation. For a base sequence modified by bisulfite treatment, PCR primers corresponding to regions in which a 5′-CpG-3′ base sequence is present may be constructed. For example, two kinds of primers corresponding to the methylated case and the unmethylated case may be generated. More specifically, primer pairs may thus be designed to be “methylated-specific” by including sequences complementing only unconverted 5-methylcytosines, or, on the converse, “unmethylated-specific”, complementing thymines converted from unmethylated cytosines. Methylation is determined by the ability of the specific primer to achieve amplification. When genomic DNA is modified with bisulfite and then subjected to PCR using the two kinds of primers, if DNA is methylated, then a PCR product can be made from the DNA from a primer corresponding to the methylated base sequence. In contrast, if that region of the gene is unmethylated, a PCR product can be made from the DNA based on a primer corresponding to the unmethylated base sequence. The methylation of DNA can be qualitatively analyzed, e.g., using agarose gel electrophoresis.

In some embodiments, placing the CpG pair at the 3′-end of a primer may improve the sensitivity. The initial report using MSP described sufficient sensitivity to detect methylation of 0.1% of alleles.

B. Real-Time Methylation-Specific PCR

Real-time methylation-specific PCR generally involves a real-time measurement method, such as real-time PCR, modified from methylation-specific PCR. The method may involve treating genomic DNA with bisulfite, and utilizing methylated-specific and unmethylated-specific PCR primers in combination with real-time PCR. The method may involve performing detection using a TaqMan® probe complementary to the amplified base sequence, or detection using SYBRgreen®. Generally, real-time methylation-specific PCR can quantitatively analyze DNA. A standard curve may be prepared using an in vitro methylated DNA sample, and for standardization, a gene having no 5′-CpG-3′ sequence in the base sequence may be amplified as a negative control; in this way the degree of methylation of a gene may be calculated.

The MethyLight method is an example of a method that is based on MSP, but can provide a quantitative analysis using real-time PCR (Eads et al., 2000). Methylated-specific primers are typically used, and a methylated-specific fluorescence reporter probe may also be used to anneal to the amplified region. Alternately, the primers or probe can be designed without methylation specificity, e.g., if discrimination is desired between the CpG pairs within the involved sequences. Quantification can be calculated in comparison to a methylated reference DNA. This protocol may be modified to increase the specificity of the PCR for successfully bisulfite-converted DNA by using an additional probe to bisulfite-unconverted DNA to quantify a non-specific amplification (Rand et al., 2002).

Melting-curve analysis (Mc-MSP) may also be used to quantify the amount of methylation in a DNA, and generally involves the evaluation of MSP-amplified DNA (Akey et al., 2002). This method generally involves amplifying bisulfite-converted DNA with both methylated-specific and unmethylated-specific primers, and determining the quantitative ratio of the two products by comparing the differential peaks generated in a melting-curve analysis. Some Mc-MSP methods may use both real-time quantification and melting analysis, which may be particularly useful, e.g., for sensitive detection of low-level methylation (Kristensen et al., 2008).

C. DNA Sequencing

DNA sequencing, including single molecule sequencing, such as pyrosequencing or sequencing by ligation (e.g., SOLiD™), may be used to detect the presence, absence, or amount of methylation of a gene. Such sequencing may be used to analyze bisulfite-treated DNA without the need for methylation-specific PCR (Colella et al., 2003; Tost et al., 2003). Sequencing is then employed (with or without first amplifying the sequence by PCR) to determine the bisulfite-converted sequence of specific CpG sites in the region. The ratio of C-to-T at individual sites can be determined quantitatively based on the amount of C and T incorporation during the sequence extension. Pyrosequencing, for example, may be particularly effective for high-throughput screening methods or for examining large regions of genomic DNA. In some embodiments, allele-specific primers may be used that incorporate single-nucleotide polymorphisms into the sequence of the sequencing primer (Wong et al., 2006).

D. Base-Specific Cleavage/MALDI-TOF

Base-specific cleavage/MALDI-TOF may be used to detect methylation of a gene (Ehrich et al. 2005). This method typically involves using in vitro transcription of the region of interest into RNA (e.g., by adding an RNA polymerase promoter site to the PCR primer in the initial amplification), and then cleavage of the RNA transcript at base-specific sites with RNase A. Since RNase A can cleave RNA specifically at cytosine and uracil ribonucleotides, base-specificity is achieved by adding incorporating cleavage-resistant dTTP when cytosine-specific (C-specific) cleavage is desired, and incorporating dCTP when uracil-specific (U-specific) cleavage is desired. The cleaved fragments can then be analyzed by MALDI-TOF. Bisulfite treatment can result in either introduction/removal of cleavage sites by C-to-U conversions or shift in fragment mass by G-to-A conversions in the amplified reverse strand. C-specific cleavage can cut specifically at the methylated CpG sites. By analyzing the sizes of the resulting fragments, it is possible to determine the specific pattern of DNA methylation of CpG sites within the region, rather than determining the extent of methylation of the region as a whole.

E. Methylation-Sensitive Single-Strand Conformation Analysis (MS-SSCA)

Methylation-sensitive single-strand conformation analysis (MS-SSCA) may be used to detect methylation in a gene. This method is based on the single-strand conformation polymorphism analysis (SSCA) method, which has been used for single-nucleotide polymorphism (SNP) analysis (Bianco et al., 1999). SSCA can differentiate between single-stranded DNA fragments of identical size but distinct sequence based on differential migration in non-denaturating electrophoresis. In MS-SSCA, this approach can be used to distinguish between bisulfite-treated, PCR-amplified regions containing the CpG sites of interest. Bisulfite treatment of DNA can make C-to-T conversions in most regions, which can result in high sensitivity. MS-SSCA can provide semi-quantitative analysis of the degree of DNA methylation based on the ratio of band intensities. This method may be used to evaluate most or all CpG sites in a DNA region of interest.

F. High Resolution Melting Analysis (HRM)

High-resolution melting analysis (HRM) is a real-time PCR-based technique which may be used to detect methylation, e.g., by differentiating converted from unconverted bisulfite-treated DNA (Wojdacz and Dobrovic, 2007). PCR amplicons can be analyzed directly by temperature ramping and resulting liberation of an intercalating fluorescent dye during melting. The degree of methylation, as represented by the C-to-T content in the amplicon, can be used to determine the rapidity of melting and consequent release of the dye. This method can allow for detecting methylation in a gene in a single-tube assay.

G. Methylation-Sensitive Single-Nucleotide Primer Extension (MS-SnuPE)

Methylation-sensitive single-nucleotide primer extension (MS-SnuPE) may be used to detect methylation of a gene (Gonzalgo and Jones, 1997). DNA is bisulfite-converted, and bisulfite-specific primers are annealed to the sequence up to the base pair immediately before the CpG of interest. The primer is allowed to extend one base pair into the C (or T) using DNA polymerase terminating dideoxynucleotides, and the ratio of C to T is determined quantitatively. The C:T ratio may be determined by a variety of techniques including, e.g., radioactive ddNTPs incorporation, fluorescence-based methods, pyrosequencing, matrix-assisted laser desorption ionization/time-of-flight (MALDI-TOF) mass spectrometry, or ion pair reverse-phase high-performance liquid chromatography (IP-RP-HPLC) has also been used to distinguish primer extension products (Uhlmann et al., 2002; Matin et al., 2002).

H. Detection of Differential Methylation-Methylation Sensitive Restriction Endonuclease

Detection of methylation in a gene can be accomplished, in some embodiments, by contacting a nucleic acid sample with a methylation sensitive restriction endonuclease that cleaves only unmethylated CpG sites under conditions and for a time to allow cleavage of unmethylated nucleic acid. In a separate reaction, the sample may be further contacted with an isoschizomer of the methylation sensitive restriction endonuclease that cleaves both methylated and unmethylated CpG-sites under conditions and for a time to allow cleavage of methylated nucleic acid. Specific primers may be added to the nucleic acid sample under conditions and for a time to allow nucleic acid amplification to occur. The presence of amplified product in the sample digested with methylation sensitive restriction endonuclease but absence of an amplified product in sample digested with an isoschizomer of the methylation sensitive restriction enzyme endonuclease that cleaves both methylated and unmethylated CpG-sites can indicate that methylation has occurred at the nucleic acid region being assayed. Lack of amplified product in the sample digested with methylation sensitive restriction endonuclease together with lack of an amplified product in the sample digested with an isoschizomer of the methylation sensitive restriction enzyme endonuclease that cleaves both methylated and unmethylated CpG-sites can indicate that methylation has not occurred at the nucleic acid region being assayed.

Real-time MSRE-PCR-Optimized single-step methylation sensitive restriction enzyme PCR can also be achieved using a OneStep qMethyl™ kit (available from Zymo Research Corp, Irvine, Calif.). This system can be used for the detection of locus-specific DNA methylation by selective amplification of a methylated region of DNA. This is accomplished by splitting any DNA to be tested into two parts: a “Test Reaction” and a “Reference Reaction”. DNA in the Test Reaction is digested with Methylation Sensitive Restriction Enzymes (MSREs) while DNA in the Reference Reaction is not digested. The DNA from both samples is then amplified using real-time PCR in the presence of a fluorescent dye, such as SYTO®9, and then quantified. Because of the simplicity of the method such as system can be used to examine multiple sites of potential methylation (e.g., 10, 50, 100, 1,000 or more sites) essentially simultaneously and can provide a quantitative readout of methylation at each site.

I. Microarray and DNA Chip-Based Methods

Microarray-based or DNA Chip-based methods may be used to detect methylation, e.g., in bisulfite-treated DNA (Adorján et al., 2002). An oligonucleotide microarray or DNA chip may be produced using oligonucleotide pairs targeting CpG sites of interest, e.g., with one or more primer complementary to a methylated sequence, and another primer complimentary to a C-to-U-converted unmethylated sequence. The oligonucleotides may be bisulfite-specific to prevent binding to any DNA which has been incompletely converted by bisulfite. Microarray-based methods include, e.g., the Illumina Methylation Assay. In some embodiments, a microarray or DNA chip may be configured to detect methylation in one, two, three, four, five or all six of the RAB25, NANOG, PTPN6, MGMT, GBP3 or LYST gene regions.

J. Indirect Detection

While the methodologies detailed above involve direct assessment of DNA methylation status, it will be recognized that methylation can also be indirectly assessed. For example, indirect assessment can comprise correlating methylation and gene expression levels, thus methylation status can be determined by determining the expression level of the indicated gene. Accordingly, in one embodiment a method if provided for assessing pluripotency by determining the expression level of one or more genes selected from the group consisting of RAB25, NANOG, PTPN6, MGMT, GBP3 or LYST. Any of a variety of methods well known in the art can be used for determining an expression level of gene including determining the RNA expression level (e.g., by quantitative hybridization or reverse transcription PCR) or the protein expression level (e.g., by immunoblot or ELISA)

### IV. Kits

The technology herein includes kits for evaluating presence, absence, or amount of methylation in a particular locus. For example, reagents can be provided for assessing methylation in the RAB25, NANOG, PTPN6, MGMT, GBP3 or LYST gene regions in a sample. A “kit” refers to a combination of physical elements. For example, a kit may include, for example, one or more components such as probes, including without limitation specific primers, enzymes, reaction buffers, an instruction sheet, and other elements useful to practice the technology described herein. The kits may include one or more primers, such as primers for PCR, to detect methylation of one or more of the genes as described herein. These physical elements can be arranged in any way suitable for carrying out the invention.

Kits for analyzing methylation of one or more genes may include, for example, a set of oligonucleotide probes for detecting methylation in RAB25, NANOG, PTPN6, MGMT, GBP3 or LYST. The probes can be provided on a solid support, as in an array (e.g., a microarray), or in separate containers. The kits can include a set of oligonucleotide primers useful for amplifying a set of genes described herein, such as to perform PCR analysis. Kits can include further buffers, enzymes, labeling compounds, and the like. Any of the compositions described herein may be comprised in a kit. The kit may further include water and hybridization buffer to facilitate hybridization of the two nucleic acid strands.

The components of the kits may be packaged either in aqueous media or in lyophilized form. The container means of the kits will generally include at least one vial, test tube, flask, bottle, syringe or other container means, into which a component may be placed, and preferably, suitably aliquoted (e.g., aliquated into the wells of a microtiter plate). Where there is more than one component in the kit, the kit also will generally contain a second, third or other additional container into which the additional components may be separately placed. However, various combinations of components may be comprised in a single vial. The kits of the present invention also will typically include a means for containing the nucleic acids, and any other reagent containers in close confinement for commercial sale. Such containers may include injection or blow molded plastic containers into which the desired vials are retained.

A kit will also include instructions for employing the kit components as well the use of any other reagent not included in the kit. Instructions may include variations that can be implemented. It is contemplated that such reagents are embodiments of kits of the invention. Such kits, however, are not limited to the particular items identified above and may include any reagent used for the manipulation or characterization of the methylation of a gene.

The container means of the kits will generally include at least one vial, test tube, flask, bottle, or other container means, into which a component may be placed, and preferably, suitably aliquoted. Where there is more than one component in the kit, the kit also will generally contain additional containers into which the additional components may be separately placed. However, various combinations of components may be comprised in a container. The kits of the present invention also will typically include a means for packaging the component containers in close confinement for commercial sale. Such packaging may include injection or blow-molded plastic containers into which the desired component containers are retained.

### V. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Identification and Validation of Epigenetic Markers of Pluripotency

A select group of potential epigenetic markers was screened to determine which is any could be used for quantitative assessment of cellular pluripotency. For each candidate marker a four phase study was used to determine the possible value of the marker. Evaluation criterion for each phase is listed below. Results of the evaluations are shown in Table 3 below.

### Phase I: Test for Discrimination Between Methylated (M) and Unmethylated (U) DNA

Studies were undertaken to determine:
(1) Whether a difference between the unmethylated DNA standard and the completely methylated DNA standard was evident. 4 or more CT values between the standards was the cut off used. Theoretically these values should be 0% for the unmethylated and 100% for the methylated standards—however, 10-15% methylation error for both standards was allowed. or
(2) Whether a melt curve showed a single peak.

If neither (1) not (2) was met, then the marker failed the phase I evaluation. Results of the studies are shown in Table 3.

### Phase II: Test for Correlation Between Actual and Experimental Methylation Proportion

For these studies samples with known levels of methylation were assayed to determine if the actual proportion of methylation could be experimentally determined. Correlation coefficient of actual methylation percentages and calculated methylation percentages must be 0.95 or better for 0, 25, 50, 75, and 100% methylation percentages. 10% percent methylation error is allotted for each of the methylation percentages if R²=<0.95 assay is failed. Formula to calculate methylation percentages=ΔCt=Average Ct value of the reference reaction−Average Ct value of the test reaction 2^{-ΔCt}*100=Methylation percentage of the unmethylated DNA standard. 100% minus the unmethylated DNA standard=methylation percentage of the methylated standard. Example results for Nanog are shown in Table 1 below. Results for all other candidate marker are summarized in Table 3.

TABLE 1
Example phase II methylation correlation data for NANOG

| NANOG (P10) | Test Ave. | std. dev. | Ref. Ave. | std. dev. | delta CT | % meth |
|---|---|---|---|---|---|---|
| 0 | 31.59262 | 0.110917 | 26.72438 | 0.132452 | 4.86824 | 3.423843 |
| 25 | 28.42397 | 0.151887 | 26.74299 | 0.062052 | 1.680981 | 31.18706 |
| 50 | 27.2626 | 0.119561 | 26.48832 | 0.094205 | 0.774279 | 58.46809 |
| 75 | 26.54072 | 0.395928 | 26.3252 | 0.130024 | 0.215518 | 86.12367 |
| 100 | 26.54265 | 0.08861 | 26.67647 | 0.031793 | −0.13382 | 109.7193 |

### Phase III: Biological Significance of the Marker

Biological samples (N=5 or more) were run with this assay. If the biological tests failed to show significant difference between stem cells and somatic cells then the marker failed. Example studies results for NANOG are shown below in Table 2. The qMethyl methylation percentages for NANOG confirm a low level of methylation in embryonic stem cell lines, and its high level of methylation in differentiated cell lines (liver). The methylation standards are also within the levels predicted, therefore this assay is successful. A summary of results with other candidate markers are shown below in Table 3.

TABLE 2
Nanog methylation discriminates between differentiated and pluripotent cells.

| NANOG (P10) | Test Ave. | std. dev. | Ref. Ave. | std. dev. | delta CT | % meth |
|---|---|---|---|---|---|---|
| ES-017 | 34.15995 | 0.363549 | 25.19535 | 0.062627 | 8.964605 | 0.200164 |
| ES-035 | 34.89476 | 0.436758 | 27.01399 | 0.123351 | 7.880771 | 0.424279 |
| ES-049 | 33.98351 | 0.158389 | 25.79875 | 0.35527 | 8.184759 | 0.34367 |
| ES-053 | 36.75102 | 1.048665 | 28.05197 | 0.086582 | 8.699044 | 0.240617 |
| liver | 28.16102 | 0.137496 | 25.74667 | 0.14771 | 2.414348 | 18.75896 |
| unmeth | 31.15289 | 0.123604 | 26.29147 | 0.569734 | 4.861417 | 3.440073 |
| meth | 25.97813 | 0.049709 | 25.99873 | 0.06693 | −0.0206 | 101.4384 |

### Phase IV: Reproducibility of Marker Results

Biological assays (above) were repeated and run on three types of thermocyclers, ABI, Bio-Rad, and the Roche LightCycler to confirm results. Markers that lacked reproducibility failed the Phase IV evaluation. A summary of results for candidate markers is shown below in Table 3.

TABLE 3
Evaluation of candidate epigenetic markers.

| | | | | | | | | | | Phase |
|---|---|---|---|---|---|---|---|---|---|---|
| | | | | | | | | | | IV-Diff. |
| | | | | | Ampli- | | | | Phase | machine |
| | | | | | con | | Phase I- | Phase | III- | and |
| | Sequence | | | GC % | length | MSRE | U vs M | II-Std | Cell | beta- |
| Gene | Name | Primer sequence | Tm | content | (bp) | sites | initial | Curve | lines | test |
| GBP3 | GBP3 (P1)- | TGTGTCTCACATCAGGCTCAG | 56.4 | 52.4 | 152 | 2 | Pass | Pass | Pass | Pass |
| | F | (SEQ ID NO: 9) | | | | | | | | final |
| | GBP3 (P1)- | ATTGCTTCCTCTCCAGCTCT | 55.5 | 52.4 | | | | | | primer |
| | R | (SEQ ID NO: 10) | | | | | | | | on panel |
| GATAD2B | GATAD2B | CAGCTGAACTGAGCCATACC | 55.6 | 55 | 280 | 1 | Pass | Fail | | |
| | (P1)-F | (SEQ ID NO: 19) | | | | | | | | |
| | GATAD2B | TGATGTCCTGGCAAAGCGAC | 58.1 | 55 | | | | | | |
| | (P1)-R | (SEQ ID NO: 20) | | | | | | | | |
| SP100 | SP100 (P1)- | AGGAGCAGGACAGCTGTTGG | 59.7 | 60 | 321 | 3 | Pass | Fail | | |
| | F | (SEQ ID NO: 21) | | | | | | | | |
| | SP100 (P1)- | AGACCCTGAGCCAGTCATGG | 59.1 | 60 | | | | | | |
| | R | (SEQ ID NO: 22) | | | | | | | | |
| EPHA1 | EPHA1 (P2)- | TAGGAATGCCGGCTTCTGCA | 59.2 | 55 | 238 | 10 | Pass | Fail | | |
| | F | (SEQ ID NO: 23) | | | | | | * too | | |
| | EPHA1 (P3)- | TCTCCTAGTCCCTTGCAACCTG | 58.4 | 54.5 | | | | many | | |
| | R | (SEQ ID NO: 24) | | | | | | tries | | |
| OCT(4) | OCT4 (P8)- | ACACCTCAGAGCCTGGCCCAA | 63.2 | 61.9 | 230 | 4 | Pass | Fail | | |
| | F | (SEQ ID NO: 25) | | | | | | | | |
| | OCT4 (P7)- | ATGAGGGCTTGCGAAGGGACT | 60.8 | 57.1 | | | | | | |
| | R | (SEQ ID NO: 26) | | | | | | | | |
| NANOG | NANOG | GTTGGAAACGTGGTGAACCT | 55.3 | 50 | 286 | 2 | Pass | Pass | Pass | Pass |
| | (P10)-F | (SEQ ID NO: 3) | | | | | | | | final |
| | NANOG | AACATGAGGCAACCAGCTCA | 56.8 | 50 | | | | | | primer |
| | (P10)-R | (SEQ ID NO: 4) | | | | | | | | on panel |
| NANOG | NANOG | GTTGGAAACGTGGTGAACCT | 55.3 | 50 | 264 | 2 | Pass | Fail | | |
| | (P10)-F | (SEQ ID NO: 27) | | | | | | | | |
| | NANOG | CCAGCAGAACGTTAAAATCCT | 53.1 | 42.9 | | | | | | |
| | (P11)-R | (SEQ ID NO: 28) | | | | | | | | |
| LYST | LYST (P2)- | TGCACCACAGAAACCTCGGAA | 59.1 | 52.4 | 152 | 1 | Pass | Pass | Pass | Pass |
| | F | (SEQ ID NO: 11) | | | | | | | | final |
| | LYST (P2)- | AAAGCAACGCAAGGGCTTCT | 58.1 | 50 | | | | | | primer |
| | R | (SEQ ID NO: 12) | | | | | | | | on panel |
| PTPN6 | PTPN6-IDT | AGAGATGCTGTCCCGTGGGT | 60.8 | 60 | 152 | 4 | Pass | Pass | Pass | Pass |
| | (1) | (SEQ ID NO: 5) | | | | | | | | final |
| | PTPN6-IDT | TAGGGACCGGAAACAGGCGCA | 63.4 | 61.9 | | | | | | primer |
| | (2) | (SEQ ID NO: 6) | | | | | | | | on panel |
| SALL4 | SALL4 (P1)- | GAGAGGAGGAGASCCTCTCCT | 56.9 | 60 | 259 | 7 | Fail (all | | | |
| | F | (SEQ ID NO: 29) | | | | | 5 | | | |
| | SALL4 (P2)- | ACATCAACTCGGAGGAGGAC | 56.3 | 55 | | | designs) | | | |
| | R | (SEQ ID NO: 30) | | | | | drop | | | |
| | | | | | | | from list | | | |
| RAB25 | RAB25 (P4)- | AGCCCAGCAATGCACACTCAG | 60.5 | 57.1 | 258 | 1 | Pass | Pass | Pass | Pass |
| | F | (SEQ ID NO: 1) | | | | | | | | final |
| | RAB25 (P1) | AGGATCAGCTCTGGCAGGTGG | 61.4 | 61.9 | | | | | | primer |
| | R | (SEQ ID NO: 2) | | | | | | | | on panel |
| Rex1 | Rex1 (P1)-F | TCTGGGCTCTGGAGGCGCT | 63.6 | 64.8 | 185 | 10 | Pass | Fail | | |
| | | (SEQ ID NO: 31) | | | | | | | | |
| | Rexl (P1)- | GCGTTCACTCTCTGCCCGGA | 61.8 | 65 | | | | | | |
| | R | (SEQ ID NO: 32) | | | | | | | | |
| MGMT | MGMT (P1)- | GCTTTCAGGACCACTCGGGCA | 61.9 | 61.9 | 156 | 4 | Pass | Pass | Pass | Pass |
| | F | (SEQ ID NO: 7) | | | | | | | | final |
| | MGMT (P1)- | ATGGCCCTTCGGCCGGTACAA | 63.6 | 61.9 | | | | | | primer |
| | R | (SEQ ID NO: 8) | | | | | | | | on panel |
| SLCSA8 | SLC5A8 | TGAAGCTAGCGGTGAGGGACA | 60.4 | 57.1 | 165 | 2 | Pass | Fail | Fail | |
| | (P2)-F | (SEQ ID NO: 33) | | | | | | | | |
| | SLC5A8 | TGGTGTGGGACTACGTGGTGT | 60.6 | 57.1 | | | | | | |
| | (P2)-R | (SEQ ID NO: 34) | | | | | | | | |
| SLCSA8 | SLC5A8 | SLC5A8 (P3)-F | 64.3 | 61.9 | 236 | 7 | Pass | Fail | | |
| | (P3)-F | | | | | | (on | (Close) | | |
| | SLC5A8 | SLC5A8 (P1)-R | 60.8 | 57.1 | | | hold) | (R2 = | | |
| | (P2)-R | | | | | | | 0.88) | | |
| PYCARD | PYCARD | AGGCGCTTCCTTACTACACC | 56.8 | 55 | 350 | 4 | Pass | Pass | Fail | |
| | (P2)-F | (SEQ ID NO: 35) | | | | | | | | |
| | PYCARD | ATTGAGGGAGCTTCACGCTT | 56.5 | 50 | | | | | | |
| | (P1)-R | (SEQ ID NO: 36) | | | | | | | | |

### Example 2

### Epigenetic Assessment of Pluripotency

Stem Cell Gene Regions for the OneStep qMethyl™ Analysis

Markers identified in Example 1 were finalized in to a panel for further testing and assessment. Stem cell gene regions of RAB25, NANOG, PTPN6, MGMT, GBP3, and LYST were converted to their genomic sequence and searched in the University of California Santa Cruz (UCSC) genome data base. The regions were searched for specific methylation sensitive restriction enzymes sites in order to be utilized in the OneStep qMethyl™ system (see, e.g., PCT Publn. WO 2011/109529, incorporated herein by reference). Unique primers were designed for real-time PCR using the OneStep qMethyl™ System for each of the six gene regions. Regions contained anywhere from 1 to 4 Methylation Sensitive Restriction Enzyme sites per amplicon. The amplicon sizes ranged from 286 bp to 152 bp. Primers were reconstituted according to their nm concentration to a final concentration of 100 μM. Primer and amplicon sequences are found in Tables 4 and 5.

TABLE 4
Primer sequences, melting temperature, GC content, amplicon length,
and number of MSRE sites for RAB25, NANOG, PTPN6, MGMT, GBP3, and LYST.

| | | | | | Amplicon | |
|---|---|---|---|---|---|---|
| | Sequence | | | GC % | length | MSRE |
| Gene | Name | Primer sequence | Tm | content | (bp) | sites |
| RAB25 | RAB25- | AGCCCAGCAATGCACACTCAG | 60.5 | 57.1 | 258 | 1 |
| | F | (SEQ ID NO: 1) | | | | |
| | RAB25- | AGGATCAGCTCTGGCAGGTGG | 61.4 | 61.9 | | |
| | R | (SEQ ID NO: 2) | | | | |
| NANOG | NANOG- | GTTGGAAACGTGGTGAACCT | 55.3 | 50 | 286 | 2 |
| | F | (SEQ ID NO: 3) | | | | |
| | NANOG- | AACATGAGGCAACCAGCTCA | 56.8 | 50 | | |
| | R | (SEQ ID NO: 4) | | | | |
| PTPN6 | PTPN6- | AGAGATGCTGTCCCGTGGGT | 60.8 | 60 | 152 | 4 |
| | F | (SEQ ID NO: 5) | | | | |
| | PTPN6- | TAGGGACCGGAAACAGGCGCA | 63.4 | 61.9 | | |
| | R | (SEQ ID NO: 6) | | | | |
| MGMT | MGMT- | GCTTTCAGGACCACTCGGGCA | 61.9 | 61.9 | 156 | 4 |
| | F | (SEQ ID NO: 7) | | | | |
| | MGMT- | ATGGCCCTTCGGCCGGTACAA | 63.6 | 61.9 | | |
| | R | (SEQ ID NO: 8) | | | | |
| GBP3 | GBP3-F | TGTGTCTCACATCAGGCTCAG | 56.4 | 52.4 | 152 | 2 |
| | | (SEQ ID NO: 9) | | | | |
| | GBP3- | ATTGCTTCCTCTCCAGCTCT | 55.5 | 52.4 | | |
| | R | (SEQ ID NO: 10) | | | | |
| LYST | LYST-F | TGCACCACAGAAACCTCGGAA | 59.1 | 52.4 | 152 | 1 |
| | | (SEQ ID NO: 11) | | | | |
| | LYST- | AAAGCAACGCAAGGGCTTCT | 58.1 | 50 | | |
| | R | (SEQ ID NO: 12) | | | | |

TABLE 5
Amplicon sequences and chromosomal locations for RAB25, NANOG,
PTPN6, MGMT, GBP3, and LYST from the UCSC Genome Browser GRCh37/hg19
Assembly.

RAB25 chr1: 156, 030, 750-156, 031, 007 (SEQ ID NO: 13)
AGCCCAGCAATGCACACTCAGCCCTCAGTGGGCTGTCTCTGAAGGTCCTGTCCCTTTTTCGCT
TCCCCCCCGCTGGAGCTGCTTCTCCCGCTTGCGGGAGCCCAGGCTGAGAGCAGACACCCAAC
CTGTCGAACCTGTCTGACGTCATCATCTCTCCACCCACCTGGGCCCCAGGTCTCCAGCCACC
CCGCTCTTCCTGTTCTCAGCTTCCGTCCTCTCTGCTTCCTTACAGCACCCCCACCTGCCAGAG
CTGATCCT
NANOG chr12: 7, 941, 772-7, 942, 057 (SEQ ID NO: 14)
GTTGGAAACGTGGTGAACCTAGAAGTATTTGTTGCTGGGTTTGTCTTCAGGTTCTGTT
GCTCGGTTTTCTAGTTCCCCACCTAGTCTGGGTTACTCTGCAGCTACTTTTGCATTACAATGG
CCTTGGTGAGACTGGTAGACGGGATTAACTGAGAATTCACAAGGGTGGGTCAGTAGGGGGT
GTGCCCGCCAGGAGGGGTGGGTCTAA
GGTGATAGAGCCTTCATTATAAATCTAGAGACTCCAGGATTTTAACGTTCTGCTGGACTGAG
CTGGTTGCCTCATGTT
PTPN6 chr12: 7, 055, 885-7, 056, 036 (SEQ ID NO: 15)
AGAGATGCTGTCCCGTGGGTAAGTCCCGGGCACCATCGGGGTCCCAGTCTCCTGTTAGTTTT
GGAGGGAGGGAGGGCTTTGTTGATGCTCACTCCGACGTGTGTGAACGTGAGTGCGATCTGC
CGCTGCCCTGCGCCTGTTTCCGGTCCCTA
MGMT chr10: 131, 265, 028-131, 265, 183 (SEQ ID NO: 16)
GCTTTCAGGACCACTCGGGCACGTGGCAGGTCGCTTGCACGCCCGCGGACTATCCCTGTGA
CAGGAAAAGG
TACGGGCCATTTGGCAAACTAAGGCACAGAGCCTCAGGCGGAAGCTGGGAAGGCGCCGCCC
GGCTTGTACCGGCCGAAGGGCCAT
GBP3 chr1: 89, 488, 203-89, 488, 354 (SEQ ID NO: 17)
TGTGTCTCACATCAGGCTCAGCTGCAGCCTAATTTGGTCCTGGTCATTTTTAAGAAAATGAAC
TGACTTATAAATTCCTTCCCATCCTTGCCACAACGTTATAGGCTCCACGTCCCTGAGCTGAG
GTACTTCAGAGCTGGAGAGGAAGCAAT
LYST chr1: 236, 046, 796-236, 046, 948 (SEQ ID NO: 18)
TGCACCACAGAAACCTCGGAATACAACTTTCCCACGTAAGAATGAATAAACACTGAAAGAG
GCCAAAACCCCAAACACTCTGGTATGAGGACTGCTCTTCTCAAAGCCAAAAGGTCATTGGGA
TGGCTTCTTAGAAGCCCTTGCGTTGCTTTT
Primers sequences are underlined. MSRE sites are shown in bold.

### Human Embryonic Stem Cells Lines

Genomic DNA was extracted from Human embryonic stem cells lines ESI-017, ESI-035, ESI-049, ESI-051, ESI-053, (BioTime) using the Quick gDNA Mininprep kit (Zymo Research). Genomic Liver DNA was also extracted from frozen liver tissue to serve as the differentiated cells in the procedure using the Quick gDNA Miniprep kit (Zymo Research).

### Experimental Set-Up

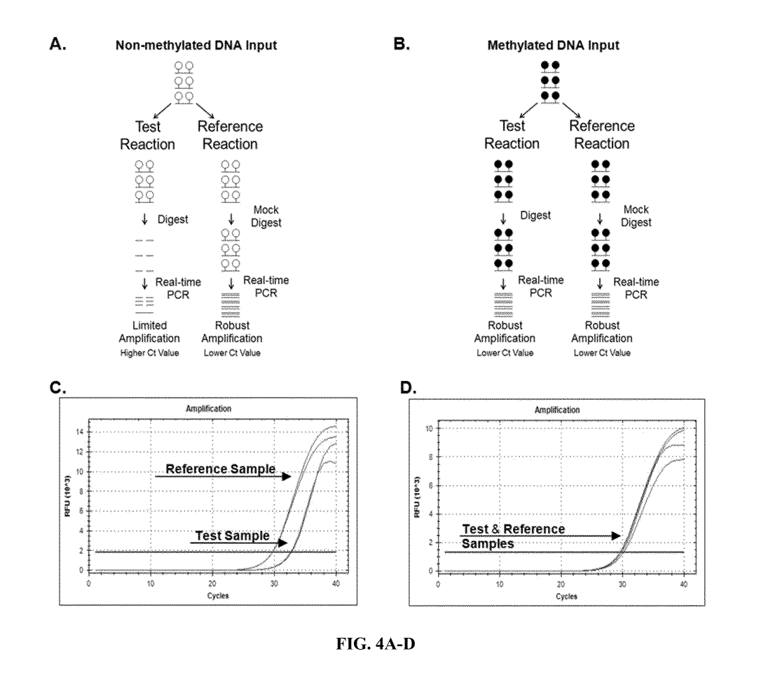

The OneStep qMethyl™ Panel can be used for the calculation of region-specific DNA methylation percentage via the selective amplification of methylated cytosines in CpG dinucleotide context. This is accomplished by splitting any DNA to be tested into two parts: a “Test Reaction” and a “Reference Reaction” (see FIG. 4). DNA in the Test Reaction is digested with Methylation Sensitive Enzymes (MSREs) while DNA in the Reference Reaction is not (or is treated with inactivated enzyme). The DNA from both samples is then amplified using real-time PCR in the presence of SYTO® 9 fluorescent dye and then quantitated. Cycle threshold (Ct) values for Test and Reference DNA samples will vary depending on methylation status, with large Ct differences most characteristic of non-methylated DNA.

Input DNA processed with the OneStep qMethyl™ procedure should be high quality for use in restriction enzyme (i.e., MSRE) digestion. For example, a nucleic acid clean-up protocol can be used to further remove impurities (e.g., Genomic DNA Clean & Concentrator™ (Cat. Nos. D4010, D4011, Zymo Research Corp.)). For Formalin-Fixed, Paraffin-Embedded (FFPE) samples, purification using the ZR FFPE DNA MiniPrep™ (Cat Nos. D3065, D3066, Zymo Research Corp.) provides, high quality, intact dsDNA.

Each reaction mixture for the OneStep qMethyl™ procedure (see below) is optimized for 20 ng input DNA. For each sample, 5 μl DNA (4 ng/μl) is added to bring the final reaction volume to 20 μl (i.e., 1 ng/μl final concentration). Input DNA is diluted in water or “modified” TE containing a low concentration of EDTA (e.g., 10 mM Tris pH 8.0-8.5. 0.1 mM EDTA).

Precise Ct value determination is critical for accurate quantification of DNA methylation by real-time PCR using the OneStep qMethyl™ protocol. Therefore, it may be preferred to set up each Test Sample and Reference Sample measurement in duplicate to ensure accurate, non-biased data collection.

The OneStep qMethyl™ Panel can be applied to a variety of applications including: screening for potential epigenetic biomarkers “epimarkers” in multiple samples, indication of pluripotency in stem cell populations, basic research and profiling, investigating environmental and dietary effects, and much more.

### Studies and Results

### Significance of the Epigenetic Markers

In order to test the effectiveness of gene regions of RAB25, NANOG, PTPN6, MGMT, GBP3, and LYST as epigenetic biomarkers for pluripotency in embryonic human stem cells, the five human embryonic cell lines, differentiated liver cells, and non-methylated and methylated standards were run in triplicates for both the test and reference values for each of the six genes using the OneStep qMethyl™ Human Pluripotent Stem Cell Panel I protocol. All samples were run on a 96 well plate in the Applied BioSystems™ 7500 Real-time thermocycler which was set to acquire data on the SYBR® green channel. The passive reference dye was set to none. The starting amount of DNA input for both test and reference reactions was 20 ng total of genomic DNA. Ct values were collected from the real-time PCR run and converted to methylation percentages based upon the −ΔCT method detailed above and in Examples 3-4 in the OneStep qMethyl™ Human Pluripotent Stem Cell Panel I protocol (Zymo Research).

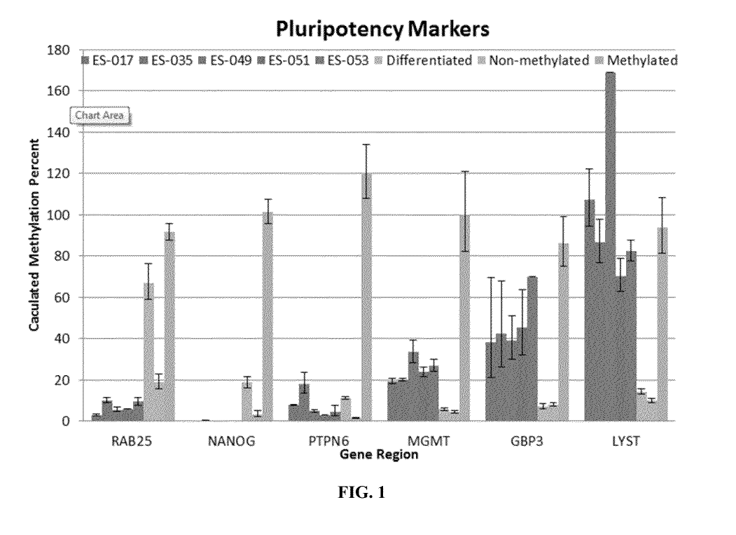

The set of five embryonic stem cell lines ESI-017, ESI-035, ESI-049, ESI-051, ESI-053, (BioTime) showed low levels of methylation percentages in the gene regions of RAB25, NANOG, and PTPN6, and high levels of methylation in the gene regions of MGMT, GBP3, and LYST using the OneStep qMethyl™ Panel Human Pluripotent Stem Cell Panel I (FIG. 1). These results were consistent with the reported literature for other human embryonic stem cell lines. Conversely the differentiated cells isolated from liver DNA showed the opposite pattern of methylation percentages, being relatively high in RAB25, NANOG, and PTPN6, and low in MGMT, GBP3, and LYST. The non-methylated DNA standard (Zymo Research) was consistently low (<20%) for all gene regions assayed, while the methylated DNA standard was consistently high (>85%) for all gene regions assayed.

A correlation between the calculated percentages of methylation and the actual methylation percentages was completed using mixtures of methylated and non-methylated DNA standards at 0, 25, 50, 75 and 100% methylation. Each methylation percentage was tested in triplicates in both the test and reference reactions for all of the six genes. The OneStep qMethyl™ Human Pluripotent Stem Cell Panel I protocol was followed as detailed in Examples 3-4. The Ct values were converted to methylation percentages and a linear correlation was done between the actual and calculated methylation percentages.

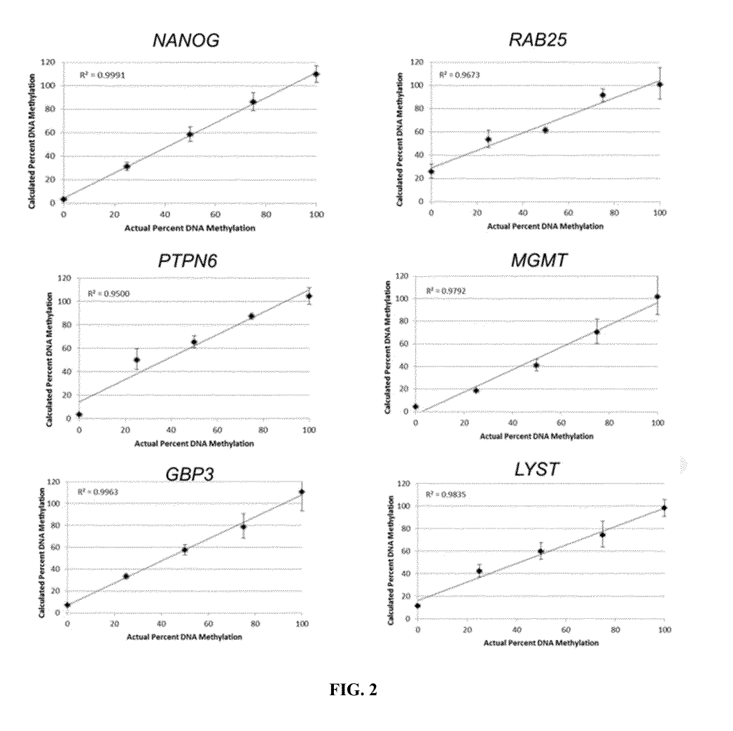

Correlation coefficients for each of the six gene assays had R² values of >0.95 (FIG. 2), indicating that the calculated methylated percentages determined using the OneStep qMethyl™ panel protocol were well correlated with the actual methylation percentages. The ability to distinguish between different methylation percentages of 0, 25, 50, 75, and 100% showed that the assay for each of the six genes is semi-quantitative.

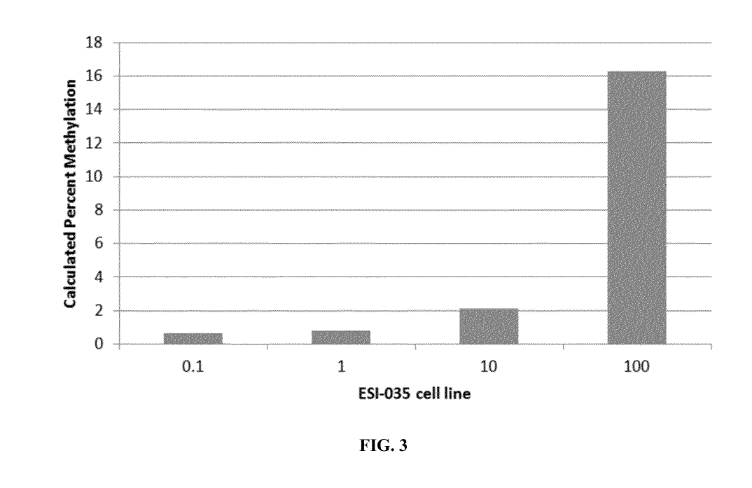

The lowest amount of differentiated DNA in a population of embryonic stem cell DNA was determined to test for assay sensitivity. Differentiated DNA was spiked into stem cell DNA at 0, 0.1, 1, 10, and 100%. The test for assay sensitivity showed that as little as 1% of differentiated DNA could be detected in a mixture of 99% embryonic stem cell DNA using the OneStep qMethyl™ Panel Human Pluripotent Stem Cell Panel protocol (FIG. 3).

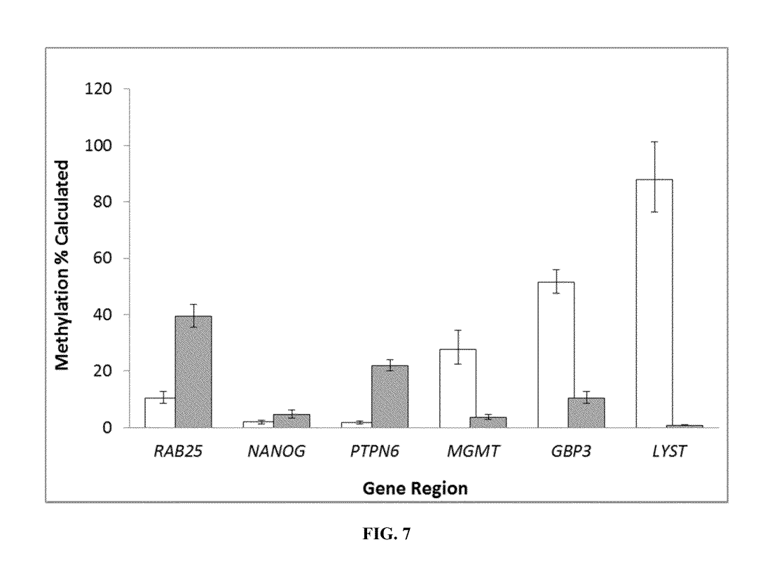

Genomic DNA from the embryonic stem cell line ESI-035 (BioTime) was also tested against genomic DNA isolated from normal human adult dermal fibroblast cells (Promocell C-14031) using the protocol for indicating pluripotency from the OneStep qMethyl™ Panel that was done previously with genomic liver DNA. The embryonic stem cell line showed low levels of methylation percentages in the gene regions of RAB25, NANOG, and PTPN6, and high levels of methylation in the gene regions of MGMT, GBP3, and LYST using the OneStep qMethyl™ Panel Human Pluripotent Stem Cell Panel I (FIG. 7). Conversely, the differentiated DNA isolated from normal human adult dermal fibroblast cells showed the opposite pattern of methylation percentages, being relatively higher in RAB25, NANOG, and PTPN6, than the embryonic stem cells, and lower in MGMT, GBP3, and LYST. These results show that the methylation percentages of these gene regions can effectively distinguish a pluripotency pattern in embryonic stem cells that is distinctly different from that of differentiated human fibroblasts. Given the extensive use of human fibroblasts for stem cell reprogramming, these results indicated that the panel could be used be to distinguish a pluripotency pattern in induced pluripotent stem cell lines (hiPSC). The panel's utility for indicating pluripotency in hiPSC lines reprogrammed from human fibroblasts has been confirmed.

### Example 3

### Example Reaction Set-Up and Analysis A

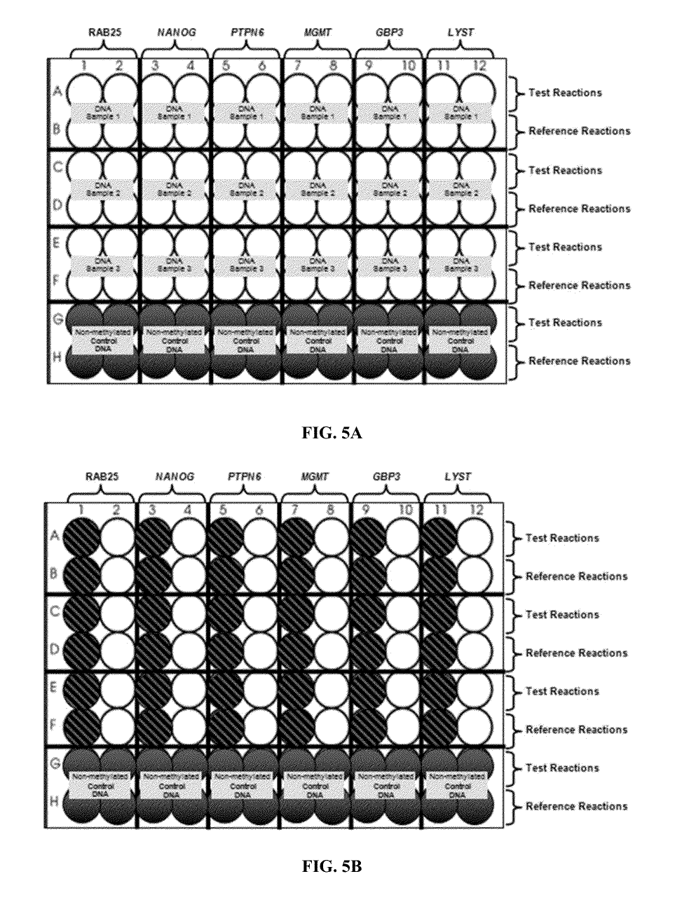

A 96-well plate is arranged with reaction components for MSRE digestions (or undigested control) and PCR. An example plate arrangement is shown in FIG. 5. After initial set-up of the plate with reaction components 5 μl (4 ng/μl) of each DNA sample to be tested is added into the appropriate well. In this example, wells G1-G12 or H1-H12 comprise non-methylated DNAs as a control. The reactions detailed here are compatible with ROX™ 2 passive reference dye for instruments requiring its usage. Thus, optionally, 1 μl ROX™ dye (50×) is added to a DNA sample prior to adding into the wells (i.e., 4 μl of DNA at 5 ng/μl+1 μl of ROX™ dye).

After formulating the reactions the plate is sealed and any bubbles removed (e.g., by centrifugation). Reaction conditions are then employed for combined digestion, and real-time amplification of DNA samples. Typically, between 40-45 cycles are used for the amplification of most DNA templates. For real-time PCR detection, the instrument is set to excitation and emission wavelengths of (˜) 465 nm and 510 nm, respectively (i.e., for SYBRgreen® or SYTO 9® dyes). Example PCR parameters are shown below:

| Step | Temperature | Time | |
|---|---|---|---|
| MSRE Digestion | 37° C. | 2 | hours |
| Initial Denaturation | 95° C. | 10 | min. |
| Denaturation* | 95° C. | 30 | sec. |
| Annealing* | 65° C. | 60 | sec |
| Extension* | 72° C. | 60 | sec |
| Final extension | 72° C. | 7 | min. |

| | | |
|---|---|---|
| Hold | 4° C. | “hold” |

*Steps repeated for 40-45 cycles.

A melt analysis to detect non-specific product and primer dimer formation can be performed after Final Extension prior to the Hold Step.

The methylation level for any amplified region can be determined using the following equation:

Percent Methylation=100×2^{-ΔCt}

where ΔCt=the average Ct values from the Test Reaction minus the average Ct values from the Reference Reaction.

The table (below) represents actual real-time PCR data generated using Human Non-methylated DNA Standard and the OneStep qMethyl™ PCR procedure and NANOG primers. (Measurements performed in duplicate).

| Ct values of Test Reaction | Ct values of Reference Reaction |
|---|---|
| 31.47 | 26.78 |
| 31.62 | 26.82 |

To determine the methylation level of the Human Non-methylated DNA Standard:

1. Calculate the average Ct value for Test Reaction and Reference Reactions.

| Average Ct value of | Average Ct value of |
|---|---|
| Test Reaction | Reference Reaction |
| 31.55 | 26.80 |

2. Determine the ΔCt by subtracting the average Ct value of the Reference Reaction from the average Ct value of the Test Reaction.

31.55−26.80=4.75

3. Plug the ΔCt into the equation: 100×2^{-ΔCt}

100×2−4.75=3.72%

Using this equation the methylation level of Human Non-methylated DNA Standard is determined to be 3.72%. Due to the low background level of methylation in Zymo's non-methylated standard cell line, the results of the calculation are within the expected limit of methylation detection. The Human Non-methylated DNA was purified from cells containing genetic knockouts of both DNMT1 and DNMT3b DNA methyltransferases and has a low level of DNA methylation (˜5%).

### Example 4

### Example Reaction Set-Up and Analysis B

DNA Methylation analysis for Pluripotency: Indicating pluripotency in stem cell lines using DNA methylation levels of RAB25, NANOG, PTPN6, MGMT, GBP3, and LYST. The following comparisons can be made using an assay of the embodiments:

1. Human DNA isolated from an embryonic stem cell line (of known pluripotent state) with an induced stem cell line (of unknown pluripotent state).

2. Human DNA isolated from an embryonic stem cell line (of questionable pluripotent state) compared to a human cell line (of a known fully differentiated state) or, an embryonic stem cell line (of a known pluripotent state).

Step I. DNA Sample Addition
1. Thaw the OneStep qMethyl™ Panel Plate on ice.
2. Quick spin plate for 1 minute to collect the contents at the bottom of the wells.
3. Carefully remove strip caps and discard. Avoid splashing to prevent potential contamination.
4. Add 5 μl (4 ng/μl) of each DNA sample into the appropriate well of the plate as indicated below. Do not add any samples to wells G1-G12 and H1-H12 as control DNAs have already been added for you. FIG. 5B illustrates the setup for triplicate sampling.
5. Seal the plate with the supplied Optical Plate Seal. Transfer to a 96-well real-time PCR instrument. Proceed with Step II

Step II. OneStep MSRE Digestion/Real-Time PCR
1. Refer to Example 3 for digestion and real-time thermal cycler conditions

Step III. Data Analysis
The table (below) represents actual real-time PCR data generated for a single gene region using the OneStep qMethyl™ Panel with differentiated DNA and human stem cell DNA1. These data are combined with those of the other 5 gene regions to assess pluripotency.

Human Differentiated DNA:

| Ct values of Test Reaction | Ct values of Reference Reaction |
|---|---|
| 25.879 | 25.500 |
| 25.934 | 25.136 |
| 25.916 | 25.361 |

Human Stem Cell DNA:

| Ct values of Test Reaction | Ct values of Reference Reaction |
|---|---|
| 29.861 | 24.758 |
| 29.628 | 24.652 |
| 29.723 | 24.659 |

1. Calculate the average and standard deviation of the Ct values for the Test and Reference of each sample.

| | Average Ct | Standard | Average Ct | Standard |
|---|---|---|---|---|
| | values of | Deviation of | values of | Deviation of |
| | Test | Test | Reference | Reference |
| Samples | Reaction | Reaction | Reaction | Reaction |
| Human | 25.910 | ±0.028 | 25.332 | ±0.184 |
| Differentiated | | | | |
| DNA | | | | |
| Human Stem | 29.737 | ±0.117 | 24.690 | ±0.060 |
| Cell DNA | | | | |

2. Determine the ΔCt by subtracting the average Ct value of the Reference Reaction from the average Ct value of the Test Reaction. Determine the standard deviation for the ΔCt using the formula for error propagation below.

$\sqrt{\begin{matrix}{( Standard Deviation Test Reaction )}^{2} + \\ {( Standard Deviation Reference Reaction )}^{2}\end{matrix}}$

| Samples | Average Ct value of Test Reaction − Average Ct value of Reference Reaction | ΔCt | $\sqrt{\begin{matrix}{( Standard Deviation Test Reaction )}^{2} + \\ {( Standard Deviation Reference Reaction )}^{2}\end{matrix}}$ | Standard Deviation ΔCt |
|---|---|---|---|---|
| Human | 25.910 − 25.332 | 0.578 | \sqrt{(0.028)² + (0.184)²} | ±0.186 |
| Differentiated | | | | |
| DNA | | | | |
| Human Stem | 29.737 − 24.690 | 5.047 | \sqrt{(0.117)² + (0.060)²} | ±0.131 |
| Cell DNA | | | | |

3. Substitute the ΔCt into the equation: 100×2−ΔCt

Human Differentiated DNA: ΔCt=0.578±0.186

100×2−0.578=66.99%

Apply the standard deviation values to the equation as well:

0.578+0.186=0.764 100×2−0.764=58.89%

0.578−0.186=0.392 100×2−0.392=76.21%

The average methylation percentage for the Human Differentiated DNA=66.99% methylation with a range of 58.89%-76.21%.

4. Repeat the same calculations for the human stem cell DNA.

Human Stem Cell DNA: ΔCt=5.047±0.131

100×2−5.047=3.02%

Apply the standard deviation values to the equation as well:

5.047+0.131=5.178 100×2−5.178=2.76%

5.047−0.131=4.916 100×2−4.916=3.31%

The average methylation percentage for the Human Stem Cell DNA=3.02% methylation with a range of 2.76%-3.31%.

Indication of Pluripotency: Stem cell lines can be successfully scored from methylation percentages calculated at RAB25, NANOG, PTPN6, MGMT, GBP3, and LYST gene regions. A positive score (+) is assigned when the calculated methylation percentage for one cell population type is significantly higher than the other. Conversely, a negative score (−) is assigned when the methylation percentage of one population is significantly lower than the other.

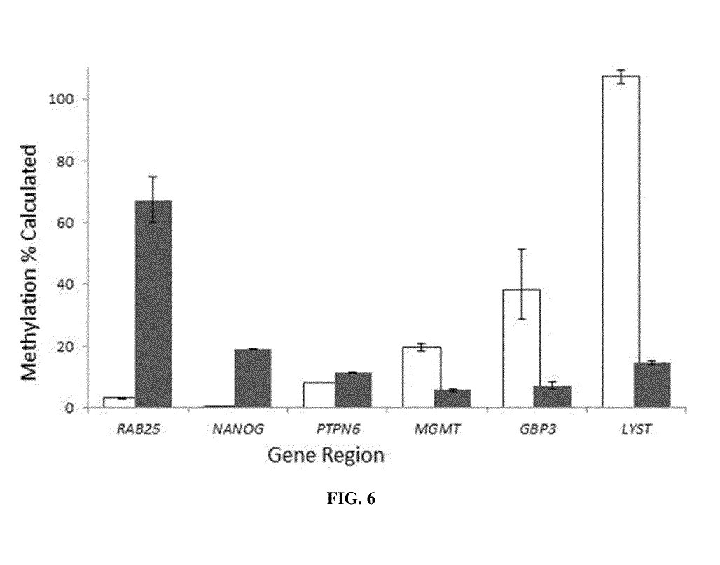

The graph in FIG. 6 illustrates the differential pattern in DNA methylation percentages for human differentiated DNA (shown as grey bars) and human stem cell DNA (shown as white bars). Likewise, data presented in FIG. 7 demonstrate that the methods detailed herein are able to easily discern methylation changes between ES cells and adult dermal fibroblasts.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 5,786,146
U.S. Patent Publn. 2010/0304992
U.S. Patent Publn. 2011/0046009\
PCT Publn. No. WO 2011/109529
Bock, C. et al., 2011. Reference maps of human ES and iPS cell variation enable high-throughput characterization of pluripotent cell lines. Cell 144, 439-452.
Muller, F.-J et al., 2011. A bioinformatic assay for pluripotency in human cells. Nat Methods 8, 315-317.
Livak and Schmittgen, 2001 Analysis of relative gene expression data using real-time quantitative PCR and the 2-ΔΔCt method. Methods 25, 402-408.
Nishino, K. et al. 2011. DNA methylation dynamics in human induced pluripotent stem cell over time. PLoS Genetics 7, (5):e1002085. doi 10.1371/journal.pgen. 1002085
Calvanese, V. et al., 2008. Cancer genes hypermethylated in human embryonic stem cells.
PLoS ONE 3, (9):e3294. doi 10.1371/journal.pone.0003294
Deb-Rinker, P. et al., Sequential DNA methylation of the Nanog and Oct-4 upstream regions in humanNT2 cells during neuronal differentiation. JBC 280(8) 6257-6260.
Li et al. 2007. A novel one cycle allele specific primer extension-Molecular beacon displacement method for DNA point mutation detection with improved specificity. Analytica Chimica Acta. 584 12-18

What is claimed is:

1. A method for assessing pluripotency in a cell population comprising:
(a) obtaining a nucleic acid sample from the cell population; and
(b) determining the DNA methylation status at 2 or more gene regions selected from the group consisting of RAB25, NANOG, PTPN6, MGMT, GBP3 and LYST,
wherein an increased level of DNA methylation relative to a reference at MGMT, GBP3 or LYST; or a decreased level of DNA methylation relative to a reference at RAB25, NANOG or PTPN6 indicates that the cell population comprises pluripotent cells.

2. The method of claim 1, wherein an increased level of DNA methylation relative to a reference at RAB25, NANOG or PTPN6; or a decreased level of DNA methylation relative to a reference at MGMT, GBP3 or LYST indicates that the cell population comprises differentiated cells.

3. The method of claim 1, wherein an increased level of DNA methylation relative to a reference at MGMT, GBP3 or LYST; or a decreased level of DNA methylation relative to a reference at RAB25, NANOG or PTPN6 indicates that the cell population comprises embryonic stem cells.

4. The method of claim 1, wherein an increased level of methylation relative to a reference at MGMT, GBP3 or LYST; or a decreased methylation relative to a reference at RAB25, NANOG or PTPN6 indicates that the cell population comprises induced pluripotent stem cells.

5. The method of claim 1, wherein the cell population is a culture of primary cells.

6. The method of claim 1, wherein the cells population is an in vivo cell population.

7. The method of claim 1, wherein the cells population comprises embryonic stem cells or induced pluripotent stem (iPS) cells.

8. The method of claim 1, further comprising:
(c) identifying the cell population as comprising pluripotent cells if the cells are determined to have an increased level of DNA methylation relative to a reference at MGMT, GBP3 or LYST; or a decreased level of DNA methylation relative to a reference at RAB25, NANOG or PTPN6; or identifying the cell population as comprising differentiated cells if the cells are determined to have an increased level of DNA methylation relative to a reference at RAB25, NANOG or PTPN6; or a decreased level of DNA methylation at MGMT, GBP3 or LYST.

9. The method of claim 8, wherein identifying comprises reporting whether the cell population comprises pluripotent cells or differentiated cells.

10. The method of claim 9, wherein the reporting comprises providing a written or electronic report.

11. The method of claim 1, further comprising using the cell population in a further protocol if the cell population is determined to comprise pluripotent cells.

12. The method of claim 2, further comprising designating the cell population for additional uses if the cell population is determined to comprise differentiated cells.

13. The method of claim 1, further defined as a method for monitoring the differentiation status in cell populations and comprising the steps of:
(a) obtaining a plurality of DNA samples from the cell populations at different time points or under different treatment conditions; and
(b) determining the DNA methylation status at 2 or more gene regions selected from the group consisting of RAB25, NANOG, PTPN6, MGMT, GBP3 and LYST in the plurality of DNA samples,
wherein an increased level of DNA methylation relative to a reference at MGMT, GBP3 or LYST; or a decreased level of DNA methylation relative to a reference at RAB25, NANOG or PTPN6 indicates that the cell population comprises greater pluripotency at a given time point or under a given treatment condition.

14. The method of claim 1, further comprising determining the DNA methylation status at least one of the gene regions selected from RAB25, NANOG and PTPN6 and at least one of the gene regions selected from MGMT, GBP3 and LYST.

15. The method of claim 14, further comprising determining the DNA methylation status at MGMT and one of the gene regions selected from RAB25, NANOG, PTPN6, GBP3 and LYST.

16. The method of claim 1, further comprising determining the DNA methylation status at 3, 4 or 5 of the gene regions.

17. The method of claim 1, further comprising determining the DNA methylation status at all six of the gene regions.

18. The method of claim 1, wherein determining the DNA methylation status at 2 or more gene regions comprises determining the DNA methylation status of a CpG position provided in SEQ ID NO: 13, 14, 15, 16, 17 or 18.

19. The method of claim 1, wherein determining a methylation status comprises performing a method selected from the group consisting of allele specific primer extension (ASPE), methylation sensitive restriction enzyme (MSRE) PCR, Real-Time MSRE, methylation specific PCR (MSP), real-time methylation specific PCR, methylation-sensitive single-strand conformation analysis (MS-SSCA), quantitative methylation specific PCR (QMSP), PCR using a methylated DNA-specific binding protein, high resolution melting analysis (HRM), methylation-sensitive single-nucleotide primer extension (MS-SnuPE), base-specific cleavage/MALDI-TOF, PCR, real-time PCR, Combined Bisulfite Restriction Analysis (COBRA), methylated DNA immunoprecipitation (MeDIP), a microarray-based method, pyrosequencing, and bisulfite sequencing.

20. The method of claim 19, wherein determining a methylation status comprises performing methylation specific PCR, real-time methylation specific PCR, QMSP, or bisulfite sequencing.

21. The method of claim 1, wherein said determining comprises treating nucleic acid in the sample with bisulfite.

22. The method of claim 1, wherein determining a methylation status comprises determining the nucleotide positions of the gene regions that comprise methylation; determining the proportion of methylated positions in the gene regions and/or quantifying the proportion of methylation at one or more CpG positions in the gene regions.

23. The method of claim 1, wherein determining a methylation status further comprises determining a hydroxymethylation status.

24. The method of claim 1, wherein the reference is a level of DNA methylation from a somatic cell or an average level of methylation from somatic cells.

25. A method of monitoring the differentiation status of cell culture comprising:
(a) exposing the cell culture to at least a first treatment; and
(b) assessing pluripotency of the cell culture in accordance with claim 1.

26. A kit comprising a sealed container comprising primers designed to amplify regions including potential DNA methylation sites in at least two gene regions selected from the group consisting of RAB25, NANOG, PTPN6, MGMT, GBP3 and LYST.

27. A method for assessing pluripotency in a cell population comprising:
(a) obtaining a DNA sample from the cell population;
(b) identifying two or more genomic amplification intervals, each interval comprising at least one CpG position within the recognition sequence of a methylation-sensitive endonuclease (MSRE), where the CpG position is subject to differential methylation during cell differentiation;
(c) amplifying the two or more genomic amplification intervals in the presence and absence of the MSRE; and
(d) quantifying the amount of amplification product to determine the proportion of DNA methylation in the two or more genomic amplification intervals, thereby assessing pluripotency in the cell population.


SEQUENCE LISTING (36 sequences)

SEQ ID NO: 1 - DNA, 21 bp, Artificial Sequence
  Synthetic primer
agcccagcaatgcacactcag

SEQ ID NO: 2 - DNA, 21 bp, Artificial Sequence
  Synthetic primer
aggatcagctctggcaggtgg

SEQ ID NO: 3 - DNA, 20 bp, Artificial Sequence
  Synthetic primer
gttggaaacgtggtgaacct

SEQ ID NO: 4 - DNA, 20 bp, Artificial Sequence
  Synthetic primer
aacatgaggcaaccagctca

SEQ ID NO: 5 - DNA, 20 bp, Artificial Sequence
  Synthetic primer
agagatgctgtcccgtgggt

SEQ ID NO: 6 - DNA, 21 bp, Artificial Sequence
  Synthetic primer
tagggaccggaaacaggcgca

SEQ ID NO: 7 - DNA, 21 bp, Artificial Sequence
  Synthetic primer
gctttcaggaccactcgggca

SEQ ID NO: 8 - DNA, 21 bp, Artificial Sequence
  Synthetic primer
atggcccttcggccggtacaa

SEQ ID NO: 9 - DNA, 21 bp, Artificial Sequence
  Synthetic primer
tgtgtctcacatcaggctcag

SEQ ID NO: 10 - DNA, 20 bp, Artificial Sequence
  Synthetic primer
attgcttcctctccagctct

SEQ ID NO: 11 - DNA, 21 bp, Artificial Sequence
  Synthetic primer
tgcaccacagaaacctcggaa

SEQ ID NO: 12 - DNA, 20 bp, Artificial Sequence
  Synthetic primer
aaagcaacgcaagggcttct

SEQ ID NO: 13 - DNA, 258 bp, Homo sapiens
agcccagcaatgcacactcagccctcagtgggctgtctctgaaggtcctgtccctttttcgcttcccccccgctggagctgcttctcccgcttgcgggagcccaggctgagagcagacacccaacctgtcgaacctgtctgacgtcatcatctctccacccacctgggccccaggtctccagccaccccgctcttcctgttctcagcttccgtcctctctgcttccttacagcacccccacctgccagagctgatcct

SEQ ID NO: 14 - DNA, 286 bp, Homo sapiens
gttggaaacgtggtgaacctagaagtatttgttgctgggtttgtcttcaggttctgttgctcggttttctagttccccacctagtctgggttactctgcagctacttttgcattacaatggccttggtgagactggtagacgggattaactgagaattcacaagggtgggtcagtagggggtgtgcccgccaggaggggtgggtctaaggtgatagagccttcattataaatctagagactccaggattttaacgttctgctggactgagctggttgcctcatgtt

SEQ ID NO: 15 - DNA, 152 bp, Homo sapiens
agagatgctgtcccgtgggtaagtcccgggcaccatcggggtcccagtctcctgttagttttggagggagggagggctttgttgatgctcactccgacgtgtgtgaacgtgagtgcgatctgccgctgccctgcgcctgtttccggtcccta

SEQ ID NO: 16 - DNA, 156 bp, Homo sapiens
gctttcaggaccactcgggcacgtggcaggtcgcttgcacgcccgcggactatccctgtgacaggaaaaggtacgggccatttggcaaactaaggcacagagcctcaggcggaagctgggaaggcgccgcccggcttgtaccggccgaagggccat

SEQ ID NO: 17 - DNA, 152 bp, Homo sapiens
tgtgtctcacatcaggctcagctgcagcctaatttggtcctggtcatttttaagaaaatgaactgacttataaattccttcccatccttgccacaacgttataggctccacgtccctgagctgaggtacttcagagctggagaggaagcaat

SEQ ID NO: 18 - DNA, 153 bp, Homo sapiens
tgcaccacagaaacctcggaatacaactttcccacgtaagaatgaataaacactgaaagaggccaaaaccccaaacactctggtatgaggactgctcttctcaaagccaaaaggtcattgggatggcttcttagaagcccttgcgttgctttt

SEQ ID NO: 19 - DNA, 20 bp, Artificial Sequence
  Synthetic primer
cagctgaactgagccatacc

SEQ ID NO: 20 - DNA, 20 bp, Artificial Sequence
  Synthetic primer
tgatgtcctggcaaagcgac

SEQ ID NO: 21 - DNA, 20 bp, Artificial Sequence
  Synthetic primer
aggagcaggacagctgttgg

SEQ ID NO: 22 - DNA, 20 bp, Artificial Sequence
  Synthetic primer
agaccctgagccagtcatgg

SEQ ID NO: 23 - DNA, 20 bp, Artificial Sequence
  Synthetic primer
taggaatgccggcttctgca

SEQ ID NO: 24 - DNA, 22 bp, Artificial Sequence
  Synthetic primer
tctcctagtcccttgcaacctg

SEQ ID NO: 25 - DNA, 21 bp, Artificial Sequence
  Synthetic primer
acacctcagagcctggcccaa

SEQ ID NO: 26 - DNA, 21 bp, Artificial Sequence
  Synthetic primer
atgagggcttgcgaagggact

SEQ ID NO: 27 - DNA, 20 bp, Artificial Sequence
  Synthetic primer
gttggaaacgtggtgaacct

SEQ ID NO: 28 - DNA, 21 bp, Artificial Sequence
  Synthetic primer
ccagcagaacgttaaaatcct

SEQ ID NO: 29 - DNA, 21 bp, Artificial Sequence
  Synthetic primer
gagaggaggagascctctcct

SEQ ID NO: 30 - DNA, 20 bp, Artificial Sequence
  Synthetic primer
acatcaactcggaggaggac

SEQ ID NO: 31 - DNA, 19 bp, Artificial Sequence
  Synthetic primer
tctgggctctggaggcgct

SEQ ID NO: 32 - DNA, 20 bp, Artificial Sequence
  Synthetic primer
gcgttcactctctgcccgga

SEQ ID NO: 33 - DNA, 21 bp, Artificial Sequence
  Synthetic primer
tgaagctagcggtgagggaca

SEQ ID NO: 34 - DNA, 21 bp, Artificial Sequence
  Synthetic primer
tggtgtgggactacgtggtgt

SEQ ID NO: 35 - DNA, 20 bp, Artificial Sequence
  Synthetic primer
aggcgcttccttactacacc

SEQ ID NO: 36 - DNA, 20 bp, Artificial Sequence
  Synthetic primer
attgagggagcttcacgctt